(19) United States
(12) Patent Application Publication
(10) Pub. No.: US 2005/0142587 A1
(43) Pub. Date: Jun. 30, 2005

(54) Mammalian genes; related reagents and methods

(75) Inventors: Constance H. Zlot, Kentfield, CA (US); Gosse Jan Adema, Groesbeek (NL); Carl Figdor, Den Bosch (NL); Joseph H. Phillips, Palo Alto, CA (US)

Correspondence Address: DNAX RESEARCH, INC.;LEGAL DEPARTMENT, 901 CALIFORNIA AVENUE, PALO ALTO, CA, 94304, US

(73) Assignee: Schering Corporation, Kenilworth, NJ (US)

(21) Appl. No.: 10/960,450

(22) Filed: Oct. 6, 2004

Related U.S. Application Data
(63) Continuation of application No. 09/713,098, filed on Nov. 14, 2000, now abandoned.
(60) Provisional application No. 60/165,438, filed on Nov. 15, 1999.

(51) Int. Cl. C12Q 1/68; G01N 33/53; C07H 21/04; C07K 14/705; C07K 16/40; C12N 9/16

(52) U.S. Cl. 435/6; 536/23.2; 435/320.1; 435/325; 530/350; 530/388.1; 435/7.1; 435/196

(57) ABSTRACT

Purified genes encoding proteins from a mammal, reagents related thereto including purified proteins, specific antibodies, and nucleic acids encoding these molecules are provided. Methods of using said reagents and diagnostic kits are also provided.

This filing is a U.S. utility patent application claiming priority to U.S. Ser. No. 60/165,438, filed Nov. 15, 1999, which is incorporated herein by reference.

### FIELD OF THE INVENTION

The present invention pertains to compositions related to proteins which function in controlling biology and physiology of mammalian cells, e.g., cells of a mammalian immune system. In particular, it provides purified genes, proteins, antibodies, and related reagents useful, e.g., to regulate activation, development, differentiation, and function of various cell types, including hematopoietic cells.

### BACKGROUND OF THE INVENTION

Recombinant DNA technology refers generally to the technique of integrating genetic information from a donor source into vectors for subsequent processing, such as through introduction into a host, whereby the transferred genetic information is copied and/or expressed in the new environment. Commonly, the genetic information exists in the form of complementary DNA (cDNA) derived from messenger RNA (mRNA) coding for a desired protein product. The carrier is frequently a plasmid having the capacity to incorporate cDNA for later replication in a host and, in some cases, actually to control expression of the cDNA and thereby direct synthesis of the encoded product in the host.

Cell lineages especially important to the immune response include two classes of lymphocytes: B-cells, which can produce and secrete immunoglobulins (proteins with the capability of recognizing and binding to foreign matter to effect its removal), and T-cells of various subsets that, secrete lymphokines and induce or suppress the B-cells and various other cells (including other T-cells) making up the immune network. These lymphocytes interact with many other cell types.

Dendritic cells (DC) are the professional antigen presenting cells (APC) of the immune system. They have the unique capability to activate naive T lymphocytes and as such play an important role in the induction of immune responses. Multiple studies have indicated that DC are superior in priming naive T cells when compared to other APC such as B cells and macrophages. Steinman (1991) Ann. Rev. Immunol. 9:271-296; Hart (1997) Blood 90:3245-3287; and Levin, et al. (1993) J. Immunol. 151:6742-6750. Recent experiments suggest that distinct DC subsets can be recognized that have strikingly different influences on the type of immune response generated in vivo. See, e.g., Rissoan, et al. (1999) Science 283, 1183-1186; Pulendran, et al. (1999) Proc. Natl. Acad. Sci. USA 96:1036-1041; Maldonado-Lopez, et al. (1999) J. Exp. Med. 189:587-592; and Smith and Fazekas de St.Groth (1999) J. Exp. Med. 189:593-598.

DC are bone marrow-derived cells, that in their immature stage are scattered throughout the body and are particularly efficient in antigen uptake through a variety of cell surface receptors specialized in capturing antigens. See Sallusto, et al. (1995) J. Exp. Med. 182:389-400. Upon inflammation, DC migrate via lymph or blood to the secondary lymphoid organs. This migration process seems to be regulated by a coordinated up- and downregulation of chemokine receptors. See Sallusto, et al. (1998) Eur. J. Immunol. 28:2760-2769; and Sozzani, et al. (1998) J. Immunol. 161:1083-1086.

Upon arrival in the T cells areas, DC are mature and fully stimulatory, well-equipped to attract and interact with naive T cells. These DC express high levels of MHC class I, MHC class II, adhesion molecules and co-stimulatory molecules, that supports the induction of primary T cell responses. Subsequently, CD40 ligation on the DC after interaction with CD4+ T helper cells leads to maximal activation of the mature DC, further increasing their capacity to stimulate naive cytotoxic T lymphocytes. See Caux, et al. (1994) J. Exp. Med. 180:1263-1272; Cella, et al. (1996) J. Exp. Med. 184:747-752; Schoenberger, et al. (1998) Nature 393:480-483; Ridge, et al. (1998) Nature 393:474-478; and Bennett, et al. (1998) Nature 393:478-480.

Besides T cells, NK cells, and macrophages, another important cell lineage is the mast cell (which has not been positively identified in all mammalian species), which is a granule-containing connective tissue cell located proximal to capillaries throughout the body. These cells are found in especially high concentrations in the lungs, skin, and gastrointestinal and genitourinary tracts. Mast cells play a central role in allergy-related disorders, particularly anaphylaxis as follows: when selected antigens crosslink one class of immunoglobulins bound to receptors on the mast cell surface, the mast cell degranulates and releases mediators, e.g., histamine, serotonin, heparin, and prostaglandins, which cause allergic reactions, e.g., anaphylaxis.

Although the role of DC in a wide variety of immunological processes has been demonstrated, the molecular mechanisms that regulate DC differentiation, migration and maturation are still poorly understood. Research to better understand and treat various immune disorders has been hampered by the general inability to maintain cells of the immune system in vitro. Immunologists have discovered that culturing these cells can be accomplished through the use of T-cell and other cell supernatants, which contain various growth factors, including many of the lymphokines.

From the foregoing, it is evident that the discovery and development of new surface antigens could contribute.to new therapies for a wide range of degenerative or abnormal conditions which directly or indirectly involve the immune system and/or hematopoietic cells. In particular, the discovery and development of lymphokines which enhance or potentiate the beneficial activities of known lymphokines would be highly advantageous. The present invention provides new compositions and related compounds, and methods for their use.

### SUMMARY OF THE INVENTION

The present invention is directed to mammalian, e.g., rodent, canine, feline, primate, proteins designated Dendritic Cell Specific Transmembrane Protein (DC-STAMP) and DNAX Surface Protein (DSP-1) and their biological activities. It includes nucleic acids coding for polypeptides themselves and methods for their production and use. The nucleic acids of the invention are characterized, in part, by their homology to complementary DNA (cDNA) sequences disclosed herein, and/or by functional assays applied to the polypeptides, which are typically encoded by these nucleic acids. Methods for modulating or intervening in the control of surface protein dependent physiology or an immune response are provided.

The present invention is based, in part, upon the discovery of novel surface proteins from dendritic cells or mast cells. In particular, it provides primate, e.g., human, sequences. Functional equivalents exhibiting significant sequence homology will be available from other. mammalian, e.g., cow, horse, rat, mouse, and non-mammalian species, e.g., warm blooded animals, including birds.

In various protein embodiments, the invention provides: a substantially pure or recombinant DC-STAMP or DSP-1 polypeptide exhibiting identity over a length of at least about 12 amino acids to SEQ ID NO: 2 or 5 or 7; a natural sequence DC-STAMP of SEQ ID NO: 2; a natural sequence DSP-1 of SEQ ID NO: 5 or 7; and a fusion protein comprising DC-STAMP or DSP-1 sequence. In certain embodiments, the segment of identity is at least about 14, 17, or 19 amino acids. In other embodiments, the DC-STAMP or DSP-1: comprises a mature sequence comprising the sequence from Tables 1 or 2; or exhibits a post-translational modification pattern distinct from natural DC-STAMP or DSP-1; or the polypeptide: is from a warm blooded animal selected from a mammal, including a primate; comprises at least one polypeptide segment of SEQ ID NO: 2 or 5 or 7; exhibits a plurality of fragments; is a natural allelic variant of DC-STAMP or DSP-1; has a length at least about 30 amino acids; exhibits at least two non-overlapping epitopes which are specific for a primate DC-STAMP or DSP-1; exhibits sequence identity over a length of at least about 20 amino acids to primate DC-STAMP or DSP-1; is glycosylated; has a molecular weight of at least 30 kD with natural glycosylation; is a synthetic polypeptide; is attached to a solid substrate; is conjugated to another chemical moiety; is a 5-fold or less substitution from natural sequence; or is a deletion or insertion variant from a natural sequence. Preferred embodiments include a composition comprising: a sterile DC-STAMP or DSP-1 polypeptide; or the DC-STAMP or DSP-1 polypeptide and a carrier, wherein the carrier is: an aqueous compound, including water, saline, and/or buffer; and/or formulated for oral, rectal, nasal, topical, or parenteral administration. In fusion protein embodiments, the protein can have: mature polypeptide sequence from Tables 1 or 2; a detection or purification tag, including a FLAG, His6, or Ig sequence; and/or sequence of another cytokine or chemokine, including Flt3 ligand.

Kit embodiments include those with a DC-STAMP or DSP-1 polypeptide, and: a compartment comprising the polypeptide; and/or instructions for use or disposal of reagents in the kit.

In binding compound embodiments, the compound may have an antigen binding site from an antibody, which specifically binds to a natural DC-STAMP or DSP-1 polypeptide, wherein: the DC-STAMP or DSP-1 is a primate protein; the binding compound is an Fv, Fab, or Fab2 fragment; the binding compound is conjugated to another chemical moiety; or the antibody: is raised against a peptide sequence of a mature polypeptide portion from Tables 1 or 2; is raised against a mature DC-STAMP or DSP-1; is raised to a purified primate DC-STAMP or DSP-1; is immunoselected; is a polyclonal antibody; binds to a denatured DC-STAMP or DSP-1; exhibits a Kd of at least 30 μM; is attached to a solid substrate, including a bead or plastic membrane; is in a sterile composition; or is detectably labeled, including a radioactive or fluorescent label. Kits containing binding compounds include those with: a compartment comprising the binding compound; and/or instructions for use or disposal of reagents in the kit. Often the kit is capable of making a qualitative or quantitative analysis. Preferred compositions will comprise: a sterile binding compound; or the binding compound and a carrier, wherein the carrier is: an aqueous compound, including water, saline, and/or buffer; and/or formulated for oral, rectal, nasal, topical, or parenteral administration.

Nucleic acid embodiments include an isolated or recombinant nucleic acid encoding a DC-STAMP or DSP-1 polypeptide or fusion protein, wherein: the DC-STAMP or DSP-1 is from a primate; and/or the nucleic acid: encodes an antigenic peptide sequence of Tables 1 or 2; encodes a plurality of antigenic peptide sequences of Tables 1 or 2; exhibits identity to a natural cDNA encoding the segment; is an expression vector; further comprises an origin of replication; is from a natural source; comprises a detectable label; comprises synthetic nucleotide sequence; is less than 6 kb, preferably less than 3 kb; is from a primate, including a human; comprises a natural full length coding sequence; is a hybridization probe for a gene encoding the DC-STAMP or DSP-1; or is a PCR primer, PCR product, or mutagenesis primer. The invention also provides a cell, tissue, or organ comprising such a recombinant nucleic acid, and preferably the cell will be: a prokaryotic cell; a eukaryotic cell; a bacterial cell; a yeast cell; an insect cell; a mammalian cell; a mouse cell; a primate cell; or a human cell.

Kit embodiments include those with such nucleic acids, and: a compartment comprising the nucleic acid; a compartment further comprising the DC-STAMP or DSP-1 protein or polypeptide; and/or instructions for use or disposal of reagents in the kit. Typically, the kit is capable of making a qualitative or quantitative analysis.

In certain embodiments, the nucleic acid:.hybridizes under wash conditions of 30° C. and less than 2M salt, or of 45° C. and/or 500 mM salt, or 55° C. and/or 150 mM salt, to SEQ ID NO: 1 or 4 or 6; or exhibits identity over a stretch of at least about 30, 55, or 75 nucleotides, to a primate DC-STAMP or DSP-1.

The invention embraces a method of modulating physiology or development of a cell or tissue culture cells comprising contacting the cell with an agonist or antagonist of a primate DC-STAMP or DSP-1. The method may be where: the contacting is in combination with an agonist or antagonist of Flt3 ligand; or the contacting is with an antagonist, including a binding composition comprising an antibody binding site which specifically binds a DC-STAMP or DSP-1.

### DETAILED DESCRIPTION OF THE PREFERRED EMBODIMENTS

All references cited herein are incorporated herein by reference to the same extent as if each individual publication or patent application was specifically and individually indicated to be incorporated by reference.

OUTLINE

| I. | General | |
|---|---|---|
| II. | Purified DC-STAMP or DSP-1 | |
| | A. | physical properties |
| | B. | biological properties |
| III. | Physical Variants | |
| | A. | sequence variants, fragments |
| | B. | post-translational variants |

| | |
|---|---|
| 1. | glycosylation |
| 2. | others |

| IV. | Functional Variants | |
|---|---|---|
| | A. | analogs, fragments |

| | |
|---|---|
| 1. | agonists |
| 2. | antagonists |

| | |
|---|---|
| B. | mimetics |

| | |
|---|---|
| 1. | protein |
| 2. | chemicals |

| | C. | species variants |
|---|---|---|
| V. | Antibodies | |
| | A. | polyclonal |
| | B. | monoclonal |
| | C. | fragments, binding compositions |
| VI. | Nucleic Acids | |
| | A. | natural isolates; methods |
| | B. | synthetic genes |
| | C. | methods to isolate |
| VII. | Making DC-STAMP or DSP-1, mimetics | |
| | A. | recombinant methods |
| | B. | synthetic methods |
| | C. | natural purification |
| VIII. | Uses | |
| | A. | diagnostic |
| | B. | therapeutic |
| IX. | Kits | |
| | A. | nucleic acid reagents |
| | B. | protein reagents |
| | C. | antibody reagents |

| | |
|---|---|
| X. | Isolating receptors for DC-STAMP or DSP-1 |

I. General

The present invention provides amino acid sequences and DNA sequences encoding various mammalian proteins which are membrane proteins, e.g., which are surface molecules which may mediate a signal between immune or other cells. See, e.g., Paul (1997) Fundamental Immunology (3d ed.) Raven Press, N.Y. The proteins, and fragments, or antagonists will be useful in physiological modulation of cells expressing a receptor or binding partner. It is likely that DC-STAMP or DSP-1 has either stimulatory or inhibitory effects on hematopoietic cells, including, e.g., lymphoid cells, such as T-cells, B-cells, natural killer (NK) cells, macrophages, dendritic cells, hematopoietic progenitors, mast cells, etc. The proteins will also be useful as antigens, e.g., immunogens, for raising antibodies to various epitopes on the protein, both linear and conformational epitopes.

Various cDNAs encoding DC-STAMP or DSP-1 were identified. The DC-STAMP was identified from cDNA libraries prepared form human monocyte-derived dendritic cells. The DSP-1 was identified from a cDNA library derived from a human HEL cell line.

Along with B lymphocytes and mononuclear phagocytes, dendritic cells (DC) are the professional antigen presenting cells (APC). DC are unique in their ability to present antigen to naive T cells, and play therefore a central role in the initiation of immune responses. Characterization of DC specific genes may help to unravel the mechanism underlying their potent antigen presenting capacity. Here is described the identification of a novel transcript, isolated by random sequencing of clones from a cDNA library prepared from monocyte-derived DC. A 2.3 kb messenger RNA is specifically expressed by DC, and not in a panel of other leukocytes or non-hematopoietic cells. In addition, no expression was detected in tissue of several human organs. The transcript encodes an approximately 470 amino acid protein, which is comprised of 7 putative transmembrane domains. This novel protein has been designated Dendritic Cell Specific Transmembrane Protein (DC-STAMP). Expression of a DC-STAMP-GFP fusion protein in 293 cells indicates that DC-STAMP is expressed at the cell surface. No sequence homology was found with another protein or multimembrane spanning receptor. DC-STAMP appears to be a novel DC-specific multimembrane spanning protein, representing a new group of transmembrane proteins.

To characterize DC at the molecular level, cDNA libraries were prepared from human monocyte-derived dendritic cells (DC) and over 250 cDNA clones were characterized by nucleotide sequence analysis. See Marland, et al. (1997) in Ricciardi-Castognoli (ed.) Dendritic Cells in Fundamental and Clinical Immunology, Vol 3, Plenum Publ. Corp. One of these cDNA clones was analyzed in further detail as it contained a unique sequence not present in the GenBank databases and its partial open reading frame (ORF) appeared to encode a putative transmembrane (TM) region. To determine the expression pattern of this novel messenger RNA, Northern blot analysis was performed using RNA from non-stimulated DC as well as RNA from a panel of freshly isolated leukocyte populations and several T, B, and monocytic cell lines. A message of 2.3 kb was specifically detected in DC but not in any of the other cell populations tested. Therefore, this novel protein was designated DC-STAMP (DC-Specific Transmembrane Protein). The finding that this RNA is enriched in the poly A⁺ RNA fraction from DC indicates that the mRNA encoding DC-STAMP is polyadenylated.

The human DC-STAMP gene will encode a membrane protein, of about 470 amino acids. See Table 1 and SEQ. ID. NO: 1 and 2. DC-STAMP exhibits structural motifs characteristic of a member of multiple membrane spanning proteins, e.g., 7 transmembrane receptors. Other notable motifs or features include asn168-thr170, asn187-ser188, and asn357-ser359 (three predicted N-linked glycosylation sites); and thr286-lys288 (potential site for phosphorylation by PKC); and lys426-ser429 and arg438-ser441 (potential sites for cAMP-dependent protein kinases).

The human DSP-1 appears to exist in two forms. One form, designated the long form, encodes a membrane protein of about 313 amino acids, and the other, designated the short form, encodes a membrane protein of about 200 amino acids. The short form seems to result from deletion of nucleotides 94-433 of the long form, and the corresponding amino acids of the protein. Both forms seem to encode type I membrane proteins, with the transmembrane segment corresponding to long form residues about leu172-gly188. Other notable motifs or features include three ITIM motifs, corresponding to long form residues leu222-leu227, val244-val251, and leu258-val263. See, e.g., Thomas (1995) J. Exp. Med. 181:1953-xx; and Lanier (1997) Immunity 6:371. Classically, the Immunoreceptor Tyrosine-based Inhibitory Motifs (ITIM) recruit intracellular tyrosine phosphatases, and the receptors provide an inhibitory signal to the cell. This suggests that the DSP-1 antigen is involved in a negative regulatory signaling pathway in the expressing cells, e.g., monocytes, T, NK, and/or mast cells. Thus, the binding partner, probably a surface receptor or soluble ligand, might inhibit monocyte, T, NK, and/or mast cell degranulation, chemotaxis, or signaling.

TABLE 1

Primate, e.g., human, DC-STAMP (SEQ ID NO: 1 and 2):

| | |
|---|---|
| ggggggtggc atttctgcat tcgaagaaga atctgagaga aacctgacgc agggagc | 57 |
| atg ggt atc tgg acc tca ggc act gat atc ttc cta agt ctt tgg gag | 105 |
| Met Gly Ile Trp Thr Ser Gly Thr Asp Ile Phe Leu Ser Leu Trp Glu | |
| 1 5 10 15 | |
| att tac gtg tct cca aga agc ccc gga tgg atg gac ttt atc cag cat | 153 |
| Ile Tyr Val Ser Pro Arg Ser Pro Gly Trp Met Asp Phe Ile Gln His | |
| 20 25 30 | |
| ttg gga gtt tgc tgt ttg gtt gct ctt att tca gtg ggc ctc ctg tct | 201 |
| Leu Gly Val Cys Cys Leu Val Ala Leu Ile Ser Val Gly Leu Leu Ser | |
| 35 40 45 | |
| gtg gcc gcc tgc tgg ttt ctg cca tca atc ata gcg gcc gct gcc tcc | 249 |
| Val Ala Ala Cys Trp Phe Leu Pro Ser Ile Ile Ala Ala Ala Ala Ser | |
| 50 55 60 | |
| tgg att atc acg tgt gtt ctg ctg tgt tgc tcc aag cat gca cqa tgt | 297 |
| Trp Ile Ile Thr Gys Val Leu Leu Cys Cys Ser Lys His Ala Arg Cys | |
| 65 70 75 80 | |
| ttt att ctt ctt gtc ttt ctc tct tgt ggc ctg cgt gaa ggc agg aat | 345 |
| Phe Ile Leu Leu Val Phe Leu Ser Cys Gly Leu Arg Glu Gly Arg Asn | |
| 85 90 95 | |
| gct ttg att gca gct ggc aca ggg atc gtc atc ttg gga cac gta gaa | 393 |
| Ala Leu Ile Ala Ala Gly Thr Gly Ile Val Ile Leu Gly His Val Glu | |
| 100 105 110 | |
| aat att ttt cac aac ttt aaa ggt ctc cta gat ggt atg act tgc aac | 441 |
| Asn Ile Phe His Asn Phe Lys Gly Leu Leu Asp Gly Met Thr Cys Asn | |
| 115 120 125 | |
| cta agg gca aag agc ttt tcc ata cat ttt cca ctt ttg aaa aaa tat | 489 |
| Leu Arg Ala Lys Ser Phe Ser Ile His Phe Pro Leu Leu Lys Lys Tyr | |
| 130 135 140 | |
| att gag gca att cag tgg att tat ggc ctt gcc act cca cta agt gta | 537 |
| Ile Glu Ala Ile Gln Trp Ile Tyr Gly Leu Ala Thr Pro Leu Ser Val | |
| 145 150 155 160 | |
| ttt gat gac ctt gtt tct tgg aac cag acc ctg gca gtc tct ctt ttc | 585 |
| Phe Asp Asp Leu Val Ser Trp Asn Gln Thr Leu Ala Val Ser Leu Phe | |
| 165 170 175 | |
| agt ccc agc cat gtc ctg gag gca cag cta aat gac agc aaa ggg gaa | 633 |
| Ser Pro Ser His Val Leu Glu Ala Gln Leu Asn Asp Ser Lys Gly Glu | |
| 180 185 190 | |
| gtc ctg agc gtc ttg tac cag atg gca aca acc aca gag gtg ttg tcc | 681 |
| Val Leu Ser Val Leu Tyr Gln Met Ala Thr Thr Thr Glu Val Leu Ser | |
| 195 200 205 | |
| tcc ctg ggt cag aag cta ctt gcc ttt gca ggg ctt tcg ctc gtc ctg | 729 |
| Ser Leu Gly Gln Lys Leu Leu Ala Phe Ala Gly Leu Ser Leu Val Leu | |
| 210 215 220 | |
| ctt ggc act ggc ctc ttc atg aag cga ttt ttg ggc cct tgt ggt tgg | 777 |
| Leu Gly Thr Gly Leu Phe Met Lys Arg Phe Leu Gly Pro Cys Gly Trp | |
| 225 230 235 240 | |
| aag tat gaa aac atc tac atc acc aga caa ttt gtt cag ttt gat gaa | 825 |
| Lys Tyr Glu Asn Ile Tyr Ile Thr Arg Gln Phe Val Gln Phe Asp Glu | |
| 245 250 255 | |
| agg gag aga cat caa cag agg ccc tgt gtg ctc ccg ctg aat aag gag | 873 |
| Arg Glu Arg His Gln Gln Arq Pro Cys Val Leu Pro Leu Asn Lys Glu | |
| 260 265 270 | |
| gaa agg agg aag tat gtc atc atc ccg act ttc tgg ccg act cct aaa | 921 |
| Glu Arg Arg Lys Tyr Val Ile Ile Pro Thr Phe Trp Pro Thr Pro Lys | |
| 275 280 285 | |
| gaa aqg aaa aac ctg ggg ctg ttt ttc ctc ccc ata ctt atc cat ctc | 969 |
| Glu Arg Lys Asn Leu Gly Leu Phe Phe Leu Pro Ile Leu Ile His Leu | |
| 290 295 300 | |
| tgc atc tgg gtg ctq ttt gca gct gta gat tat ctg ctg tat cgg ctc | 1017 |
| Cys Ile Trp Val Leu Phe Ala Ala Val Asp Tyr Leu Leu Tyr Arg Leu | |
| 305 310 315 320 | |
| att ttc tca gtg agc aag cag ttt caa agc ttg cca ggg ttt gag gtt | 1065 |
| Ile Phe Ser Val Ser Lys Gln Phe Gln Ser Leu Pro Gly Phe Glu Val | |
| 325 330 335 | |
| cac ttg aaa ctg cac gga gag aaa caa gga act caa gat att atc cat | 1113 |
| His Leu Lys Leu His Gly Glu Lys Gln Gly Thr Gln Asp Ile Ile His | |
| 340 345 350 | |
| qat tct tcc ttt aat ata tct gtg ttt gaa ccc aac tgt atc cca aaa | 1161 |
| Asp Ser Ser Phe Asn Ile Ser Val Phe Glu Pro Asn Cys Ile Pro Lys | |
| 355 360 365 | |
| cca aaa ttc ctt cta tct gag acc tgg gtt cct ctc agt gtt att ctt | 1209 |
| Pro Lys Phe Leu Leu Ser Glu Thr Trp Val Pro Leu Ser Val Ile Leu | |
| 370 375 380 | |
| ttg ata tta gtg atg ctg gga ctg ttg tcc tct atc ctt atg caa ctt | 1257 |
| Leu Ile Leu Val Met Leu Gly Leu Leu Ser Ser Ile Leu Met Gln Leu | |
| 385 390 395 400 | |
| aaa atc ctg gtg tca gca tct ttc tac ccc agc gtg gag agg aag cgc | 1305 |
| Lys Ile Leu Val Ser Ala Ser Phe Tyr Pro Ser Val Glu Arg Lys Arg | |
| 405 410 415 | |
| atc caa tat ctg cat gca aag ctg ctt aaa aaa aga tca aag cag ccg | 1353 |
| Ile Gln Tyr Leu His Ala Lys Leu Leu Lys Lys Arg Ser Lys Gln Pro | |
| 420 425 430 | |
| ctg gga gaa gtc aaa aga cgg ctg agt ctc tat ctt aca aag att cat | 1401 |
| Leu Gly Glu Val Lys Arg Arg Leu Ser Leu Tyr Leu Thr Lys Ile His | |
| 435 440 445 | |
| ttc tgg ctt cca gtc ctg aaa atg att agg aag aag caa atg gac atg | 1449 |
| Phe Trp Leu Pro Val Leu Lys Met Ile Arg Lys Lys Gln Met Asp Met | |
| 450 455 460 | |
| gca agt gca gac aag tca tgagagaccc cgactactcc tcagccacat | 1497 |
| Ala Ser Ala Asp Lys Ser | |
| 465 470 | |
| cgcaccaaca attctcttca ggtctaggat ggcagtcact attcatgccg gataatagag | 1557 |
| aactatgtga cgcagtcctc tcaggagtct gagtttacag agccaacttg cagcacctgg | 1617 |
| ttatgcctcc tttcatctca aagccaaaga gctgccaggt aaatggttat gtggtctatg | 1677 |
| ttccaaacaa accacatgat cttgcctgtg tcacaatgta acaagactct agctgggtcc | 1737 |
| cctggtgatg agtttcagca tagaataatg ttcaaggaaa agaaaacgaa aacagtttaa | 1797 |
| atctctacca cagcctcaca agcaaatgct aaggggaaca tacatgtaaa aagccagcaa | 1857 |
| actatcttca aactcttccg tccttaatgt cttccatggc tattgccccc acaatggtct | 1917 |
| cttttctccc tgctccctta ttaaagaact ctttctgaaa ccc | 1960 |

MGIWTSGTDIFLSLWEIYVSPRSPGWMDFIQHLGVCCLVALISVGLLSVAACWFLPSIIAAAASWIITC
VLLCCSKHARCFILLVFLSCGLREGRNALIAAGTGIVILGHVENIFHNFKGLLDGMTCNLRAKSFSIHF
PLLKKYIEAIQWIYGLATPLSVFDDLVSWNQTLAVSLFSPSHVLEAQLNDSKGEVLSVLYQMATTTEVL
SSLGQKLLAFAGLSLVLLGTGLFMKRFLGPCGWKYENIYITRQFVQFDERERHQQRPCVLPLNKEERRK
YVIIPTFWPTPKERKNLGLFFLPILIHLCIWVLFAAVDYLLYRLIFSVSKQFQSLPGFEVHLKLHGEKQ
GTQDIIHDSSFNISVFEPNCIPKPKFLLSETWVPLSVILLILVMLGLLSSILMQLKILVSASFYPSVER
KRIQYLHAKLLKKRSKQPLGEVKRRLSLYLTKIHFWLPVLKMIRKKQMDMASADKS
Reverse translation, e.g., nucleic acids encoding
polypeptides (N can be A, C, G, or T; SEQ ID NO: 3):
ATGGGNATHTGGACNWSNGGNACNGAYATHTTYYTNWSNYTNTGGGARATHTAYGTNWSNcCNMGNWSN
CCNGGNTGGATGGAYTTYATHCARcAYYTNGGNGTNTGYTGYYTNGTNGcNYTNATHWSNGTNGGNYTN
YTNWSNGTNGcNGCNTGYTGGTTYYTNCCNWSNATHATHGCNGCNGCNGCNWSNTGGATHATHACNTGY
GTNYTNYTNTGYTGYWSNAARCAYGCNMGNTGYTTYATHYTNYTNGTNTTYYTNWSNTGYGGNYTNMGN
GARGGNMGNAAYGCNYTNATHGCNGCNGGNACNGGNATHGTNATHYTNGGNCAYGTNGARAAYATHTTY
CAYAAYTTYAARGGNYTNYTNGAYGGNATGACNTGYAAYYTNMGNGCNAARWSNTTYWSNATHCAYTTY
CCNYTNYTNAARAARTAYATHGARGCNATHCARTGGATHTAYGGNYTNGCNACNCCNYTNWSNGTNTTY
GAYGAYYTNGTNWSNTGGAAYCARACNYTNGCNGTNWSNYTNTTYWSNCCNWSNCAYGTNYTNGARGCN
CARYTNAAYGAYWSNAARGGNGARGTNYTNWSNGTNYTNTAYCARATGGCNACNACNACNGARGTNYTN
WSNWSNYTNGGNCARAARYTNYTNGCNTTYGCNGGNYTNWSNYTNGTNYTNYTNGGNACNGGNYTNTTY
ATGAARMGNTTYYTNGGNCCNTGYGGNTGGAARTAYGARAAYATHTAYATHACNMGNCARTTYGTNCAR
TTYGAYGARMGNGARMGNCAYCARCARMGNCCNTGYGTNYTNCCNYTNAAYAARGARGARMGNMGNAAR
TAYGTNATHATHCCNACNTTYTGGCCNACNCCNAARGARMGNAARAAYYTNGGNYTNTTYTTYYTNCCN
ATHYTNATHCAYYTNTGYATHTGGGTNYTNTTYGCNGCNGTNGAYTAYYTNYTNTAYMGNYTNATHTTY
WSNGTNWSNAARCARTTYCARWSNYTNCCNGGNTTYGARGTNCAYYTNAARYTNCAYGGNGARAARCAR
GGNACNCARGAYATHATHCAYGAYWSNWSNTTYAAYATHWSNGTNTTYGARCCNAAYTGYATHCCNAAR
CCNAARTTYYTNYTNwSNGARACNTGGGTNCCNYTNWSNGTNATHYTNYTNATHYTNGTNATGYTNGGN
YTNYTNWSNWSNATHYTNATGCARYTNAARATHYTNGTNWSNGCNWSNTTYTAYCCNWSNGTNGARMGN
AARMGNATHCARTAYYTNCAYGCNAARYTNYTNAARAARMGNWSNAARCARCCNYTNGGNGARGTNAAR
MGNMGNYTNWSNYTNTAYYTNACNAARATHCAYTTYTGGYTNCCNGTNYTNAARATGATHMGNAARAAR
CARATGGAYATGGCNWSNGCNGAYAARWSN

TABLE 2
Primate, e.g., human, DSP-1 forms, both long and short forms:

DSP-1L (SEQ ID NO: 4 and 5):

| | |
|---|---|
| atg gcc tta cca gtg acc gcc ttg ctc ctg ccg cta gcc ttg ctg ctc | 48 |
| Met Ala Leu Pro Val Thr Ala Leu Leu Leu Pro Leu Ala Leu Leu Leu | |
| −20 +15 +10 | |
| cac gcc gcc agg ccg gat tac aag gac gat gac gac aag atc gat ctg | 96 |
| His Ala Ala Arg Pro Asp Tyr Lys Asp Asp Asp Asp Lys Ile Asp Leu | |
| −5 −1 1 5 10 | |
| agc aaa tgc agg acc gtg gcg ggc ccc gtg ggg gga tcc ctg agt gtg | 144 |
| Ser Lys Cys Arg Thr Val Ala Gly Pro Val Gly Gly Ser Leu Ser Val | |
| 15 20 25 | |
| cag tgt ccc tat gag aag gaa cac agg acc ctc aac aaa tac tgg tgc | 192 |
| Gln Cys Pro Tyr Glu Lys Glu His Arg Thr Leu Asn Lys Tyr Trp Cys | |
| 30 35 40 | |
| aga cca cca cag att ttc cta tgt gac aag att gtg gag acc aaa ggg | 240 |
| Arg Pro Pro Gln Ile Phe Leu Cys Asp Lys Ile Val Glu Thr Lys Gly | |
| 45 50 55 | |
| tca gca gga aaa agg aac ggc cga gtg tcc atc agg gac agt cct gca | 288 |
| Ser Ala Gly Lys Arg Asn Gly Arg Val Ser Ile Arg Asp Ser Pro Ala | |
| 60 65 70 75 | |
| aac ctc agc ttc aca gtg acc ctg gag aat ctc aca gag gag gat gca | 336 |
| Asn Leu Ser Phe Thr Val Thr Leu Glu Asn Leu Thr Glu Glu Asp Ala | |
| 80 85 90 | |
| ggc acc tac tgg tgt ggg gtg gat aca ccg tgg ctc cga gac ttt cat | 384 |
| Gly Thr Tyr Trp Cys Gly Val Asp Thr Pro Trp Leu Arg Asp Phe His | |
| 95 100 105 | |
| gat ccc gtt gtc gag gtt gag gtg tcc gtg ttc ccg gca tca acg tca | 432 |
| Asp Pro Val Val Glu Val Glu Val Ser Val Phe Pro Ala Ser Thr Ser | |
| 110 115 120 | |
| atg aca cct gca agt atc act gcg gcc aag acc tca aca atc aca act | 480 |
| Met Thr Pro Ala Ser Ile Thr Ala Ala Lys Thr Ser Thr Ile Thr Thr | |
| 125 130 135 | |
| gca ttt cca cct gta tca tcc act acc ctg ttt gca gtg ggt gcc acc | 528 |
| Ala Phe Pro Pro Val Ser Ser Thr Thr Leu Phe Ala Val Gly Ala Thr | |
| 140 145 150 155 | |
| cac agt gcc agc atc cag gag gaa act gag gag gtg gtg aac tca cag | 576 |
| His Ser Ala Ser Ile Gln Gln Glu Thr Glu Glu Val Val Asn Ser Gln | |
| 160 165 170 | |
| ctc ccg ctg ctc ctc tcc ctg ctg gca ttg ttg ctg ctt ctg ttg gtg | 624 |
| Leu Pro Leu Leu Leu Ser Leu Leu Ala Leu Leu Leu Leu Leu Leu Val | |
| 175 180 185 | |
| ggg gcc tcc ctg cta gcc tgg agg atg ttt cag aaa tgg atc aaa gct | 672 |
| Gly Ala Ser Leu Leu Ala Trp Arg Met Phe Gln Lys Trp Ile Lys Ala | |
| 190 195 200 | |
| ggt gac cat tca gag ctg tcc cag aac ccc aag cag gct gcc acg cag | 720 |
| Gly Asp His Ser Glu Leu Ser Gln Asn Pro Lys Gln Ala Ala Thr Gln | |
| 205 210 215 | |
| agt gag ctg cac tac gca aat ctg gag ctg ctg atg tgg cct ctg cag | 768 |
| Ser Glu Leu His Tyr Ala Asn Leu Glu Leu Leu Met Trp Pro Leu Gln | |
| 220 225 230 235 | |
| gaa aag cca gca cca cca agg gag gtg gag gtg gaa tac agc act gtg | 816 |
| Glu Lys Pro Ala Pro Pro Arg Glu Val Glu Val Glu Tyr Ser Thr Val | |
| 240 245 250 | |
| gcc tcc ccc agg gaa gaa ctt cac tat gcc tcg gtg gtg ttt gat tct | 864 |
| Ala Ser Pro Arg Glu Glu Leu His Tyr Ala Ser Val Val Phe Asp Ser | |
| 255 260 265 | |
| aac acc aac agg ata gct gct cag agg cct cgg gag gag gaa cca gat | 912 |
| Asn Thr Asn Arg Ile Ala Ala Gln Arg Pro Arg Glu Glu Glu Pro Asp | |
| 270 275 280 | |
| tca gat tac agt gtg ata agg aag aca tag | 942 |
| Ser Asp Tyr Ser Val Ile Arg Lys Thr | |
| 280 290 | |

MALPVTALLLPLALLLHAARPDYKDDDDKIDLSKCRTVAGPVGGSLSVQCPYEKEHRTLNKYWCRPP
QIFLCDKIVETKGSAGKRNGRVSIRDSPANLSFTVTLENLTEEDAGTYWCGVDTPWLRDFHDPVVEV
EVSVFPASTSMTPASITAAKTSTITTAFPPVSSTTLFAVGATHSASIQEETEEVVNSQLPLLLSLLA
LLLLLLVGASLLAWRMFQKWIKAGDHSELSQNPKQAATQSELHYANLELLMWPLQEKPAPPREVEVE
YSTVASPREELHYASVVFDSNTNRIAAQRPREEEPDSDYSVIRKT

DSP-1S form (SEQ ID NO: 6 and 7):

| | |
|---|---|
| atg gcc tta cca gtg acc gcc ttg ctc ctg ccg cta gcc ttg ctg ctc | 48 |
| Met Ala Leu Pro Val Thr Ala Leu Leu Leu Pro Leu Ala Leu Leu Leu | |
| −20 −15 −10 | |
| cac gcc gcc agg ccg gat tac aag gac gat gac gac aag atc gat atg | 96 |
| His Ala Ala Arg Pro Asp tyr Lys Asp Asp Asp Asp Lys Ile Asp Met | |
| −5 −1 1 5 10 | |
| aca cct gca agt atc act gcg gcc aag acc tca aca atc aca act gca | 144 |
| Thr Pro Ala Ser Ile Thr Ala Ala Lys Thr Ser Thr Ile Thr Thr Ala | |
| 15 20 25 | |
| ttt cca cct gta tca tcc act acc ctg ttt gca gtg ggt gcc acc cac | 192 |
| Phe Pro Pro Val Ser Ser Thr Thr Leu Phe Ala Val Gly Ala Thr His | |
| 30 35 40 | |
| agt gcc agc atc cag gag gaa act gag gag gtg gtg aac tca cag ctc | 240 |
| Ser Ala Ser Ile Gln Glu Glu Thr Glu Glu Val Val Asn Ser Gln Leu | |
| 45 50 55 | |
| ccg ctg ctc ctc tcc ctg ctg gca ttg ttg ctg ctt ctg ttg gtg ggg | 288 |
| Pro Leu Leu Leu Ser Leu Leu Ala Leu Leu Leu Leu Leu Leu Val Gly | |
| 60 65 70 75 | |
| gcc tcc ctg cta gcc tgg agg atg ttt cag aaa tgg atc aaa gct ggt | 336 |
| Ala Ser Leu Leu Ala trp Arg Met Phe Gln Lys trp Ile Lys Ala Gly | |
| 80 85 90 | |
| gac cat tca gag ctg tcc cag aac ccc aag cag gct gcc acg cag agt | 384 |
| Asp His Ser Glu Leu Ser Gln Asn Pro Lys Gln Ala Ala Thr Gln Ser | |
| 95 100 105 | |
| gag ctg cac tac gca aat ctg gag ctg ctg atg tgg cct ctg cag gaa | 432 |
| Glu Leu His tyr Ala Asn Leu Glu Leu Leu Met trp Pro Leu Gln Glu | |
| 110 115 120 | |
| aag cca gca cca cca agg gag gtg gag gtg gaa tac agc act gtg gcc | 480 |
| Lys Pro Ala Pro Pro Arg Glu Val Glu Val Glu tyr Ser Thr Val Ala | |
| 125 130 135 | |
| tcc ccc agg gaa gaa ctt cac tat gcc tcg gtg gtg ttt gat tct aac | 528 |
| Ser Pro Arg Glu Glu Leu His tyr Ala Ser Val Val Phe Asp Ser Asn | |
| 140 145 150 155 | |
| acc aac agg ata gct gct cag agg cct cgg gag gag gaa cca gat tca | 576 |
| Thr Asn Arg Ile Ala Ala Gln Arg Pro Arg Glu Glu Glu Pro Asp Ser | |
| 160 165 170 | |
| gat tac agt gtg ata agg aag aca tag | 603 |
| Asp tyr Ser Val Ile Arg Lys Thr | |
| 175 | |

MALPVTALLLPLALLLHAARPDYKDDDDKEDMTPASITAAKTSTITTAFPPVSSTTLFAVGATHSAS
IQEETEEVVNSQLPLLLSLLALLLLLLVGASLLAWRMFQKWIKAGDHSELSQNPKQAATQSELHYAN
LELLMWPLQEKPAPPREVEVEYSTVASPREELHYASVVFDSNTRIAAQRPREEEPDSDYSVIRKT

Alignment of long and short forms:

| | | | |
|---|---|---|---|
| DSP-1L | 1 | MALPVTALLLPLALLLHAARPDYKDDDDKIDLSKCRTVAGPVGGSLSVQC | 50 |
| DSP-1S | 1 | MALPVTALLLPLALLLHAARPDYKDDDDKID------------------- | 31 |
| | | ******************************* | |
| DSP-1L | 51 | PYEKEHRTLNKYWCRPPQIFLCDKIVETKGSAGKRNGRVSIRDSPANLSF | 100 |
| DSP-1S | 32 | -------------------------------------------------- | 31 |
| DSP-1L | 101 | TVTLENLTEEDAGTYWCGVDTPWLRDFHDPVVEVEVSVFPASTSMTPASI | 150 |
| DSP-1S | 32 | --------------------------------------------MTPASI | 37 |
| | | ****** | |
| DSP-1L | 151 | TAAKTSTITTAFPPVSSTTLFAVGATHSASIQEETEEVVNSQLPLLLSLL | 200 |
| DSP-1S | 38 | TAAKTSTITTAFPPVSSTTLFAVGATHSASIQEETEEVVNSQLPLLLSLL | 87 |
| | | ************************************************** | |
| DSP-1L | 201 | ALLLLLLVGASLLAWRMFQKWIKAGDHSELSQNPKQAATQSELHYANLEL | 250 |
| DSP-1S | 88 | ALLLLLLVGASLLAWRMFQKWIKAGDHSELSQNPKQAATQSELHYANLEL | 137 |
| | | ************************************************** | |
| DSP-1L | 251 | LMWPLQEKPAPPREVEVEYSTVASPREELHYASVVFDSNTNRIAAQRPRE | 300 |
| DSP-1S | 138 | LMWPLQEKPAPPREVEVEYSTVASPREELHYASVVFDSNTNRIAAQRPRE | 187 |
| | | ************************************************** | |
| DSP-1L | 301 | EEPDSDYSVIRKT | 313 |
| DSP-1S | 188 | EEPDSDYSVIRKT | 200 |
| | | ************* | |

Reverse translation, e.g., nucleic acids encoding polypeptides
(N can be A, C, G, or T):
long (SEQ ID NO: 8); N may be A, C, G, or T:
ATGGCNYTNCCNGTNACNGCNYTNYTNYTNCCNYTNGCNYTNYTNYTNCAYGCNGCNMGNCCNGAYT
AYAARGAYGAYGAYGAYAARATHGAYYTNWSNAARTGYMGNACNGTNGCNGGNCCNGTNGGNGGNWS
NYTNWSNGTNCARTGYCCNTAYGARAARGARCAYMGNACNYTNAAYAARTAYTGGTGYMGNCCNCCN
CARATHTTYYTNTGYGAYAARATHGTNGARACNAARGGNWSNGCNGGNAARMGNAAYGGNMGNGTNW
SNATHMGNGAYWSNCCNGCNAAYYTNWSNTTYACNGTNACNYTNGARAAYYTNACNGARGARGAYGC
NGGNACNTAYTGGTGYGGNGTNGAYACNCCNTGGYTNMGNGAYTTYCAYGAYCCNGTNGTNGARGTN
GARGTNWSNGTNTTYCCNGCNWSNACNWSNATGACNCCNGCNWSNATHACNGCNGCNAARACNWSNA
CNATHACNACNGCNTTYCCNCCNGTNWSNWSNACNACNYTNTTYGCNGTNGGNGCNACNCAYWSNGC
NWSNATHCARGARGARACNGARGARGTNGTNAAYWSNCARYTNCCNYTNYTNYTNWSNYTNYTNGCN
YTNYTNYTNYTNYTNYTNGTNGGNGCNWSNYTNYTNGCNTGGMGNATGTTYCARAARTGGATHAARG
CNGGNGAYCAYWSNGARYTNWSNCARAAYCCNAARCARGCNGCNACNCARWSNGARYTNCAYTAYGC
NAAYYTNGARYTNYTNATGTGGCCNYTNCARGARAARCCNGCNCCNCCNMGNGARGTNGARGTNGAR
TAYWSNACNGTNGCNWSNCCNMGNGARGARYTNCAYTAYGCNWSNGTNGTNTTYGAYWSNAAYACNA
AYMGNATHGCNGCNCARMGNCCNMGNGARGARGARCCNGAYWSNGAYTAYWSNGTNATHMGNAARAC
N
short (SEQ ID NO: 9); N may be A, C, G, or T:
ATGGCNYTNCCNGTNACNGCNYTNYTNYTNCCNYTNGCNYTNYTNYTNCAYGCNGCNMGNCCNGAYT
AYAARGAYGAYGAYGAYAARATHGAYATGACNCCNGCNWSNATHACNGCNGCNAARACNWSNACNAT
HACNACNGCNTTYCCNCCNGTNWSNWSNACNACNYTNTTYGCNGTNGGNGCNACNCAYWSNGCNWSN
ATHCARGARGARACNGARGARGTNGTNAAYWSNCARYTNCCNYTNYTNYTNWSNYTNYTNGCNYTNY
TNYTNYTNYTNYTNGTNGGNGCNWSNYTNYTNGCNTGGMGNATGTTYCARAARTGGATHAARGCNGG
NGAYCAYWSNGARYTNWSNCARAAYCCNAARCARGCNGCNACNCARWSNGARYTNCAYTAYGCNAAY
YTNGARYTNYTNATGTGGCCNYTNCARGARAARCCNGCNCCNCCNMGNGARGTNGARGTNGARTAYW
SNACNGTNGCNWSNCCNMGNGARGARYTNCAYTAYGCNWSNGTNGTNTTYGAYWSNAAYACNAAYMG
NATHGCNGCNCARMGNCCNMGNGARGARGARCCNGAYWSNGAYTAYWSNGTNATHMGNAARACN

The currently available methods to generate large amounts of DC in vitro allow detailed molecular analysis of DC. See Romani, et al. (1994) J. Exp. Med. 180:83-93. cDNA libraries derived from monocyte-derived DC were analyzed leading to the identification of several interesting gene products, including a novel DC-specific chemokine, DC-CK1. See Zhou and Tedder (1996) Proc. Natl. Acad. Sci. USA 93:2588-2592.

Pairwise protein sequence alignments performed between DC-STAMP and members of several 7 TM subclasses (ClustalW) showed identities below 20%, suggesting that the DC-STAMP protein represents a novel protein family. However, the structural homology of DC-STAMP to members of the superfamily of G-protein coupled (or linked) receptors (GPCR, or GPLR) suggests related function of this molecule. As a class, these receptors are integral membrane proteins characterized by amino acid sequences which contain seven hydrophobic domains. See, e.g., Ruffolo and Hollinger (eds. 1995) G-Protein Coupled Transmembrane Signaling Mechanisms CRC Press, Boca Raton, Fla.; Watson and Arkinstall (1994) The G-Protein Linked Receptor FactsBook Academic Press, San Diego, Calif.; Peroutka (ed. 1994) G Protein-Coupled Receptors CRC Press, Boca Raton, Fla.; Houslay and Milligan (1990) G-Proteins as Mediators of Cellular Signaling Processes Wiley and Sons, New York, N.Y.; and Dohlman, et al. (1991) Ann. Rev. Biochem. 60:653-688. These hydrophobic domains are predicted to represent transmembrane spanning regions of the proteins. These GPCRs are found in a wide range of organisms and are typically involved in the transmission of signals to the interior of the cell, e.g., through interaction, e.g., with heterotrimeric G-proteins. They respond to a wide and diverse range of agents including lipid analogs, amino acid derivatives, small peptides, and other molecules.

A predicted model of the structure of the DC-STAMP protein has an extracellular N-terminus, a cytoplasmic C-terminus, 3 cytoplasmic loops, and 3 extracellular loops, containing 2 consensus sequences for N-linked glycosylation on the second and one on the third extracellular loop.

The serine residues in the C-terminus of DC-STAMP are putative targets for phosphorylation. For several 7 TM proteins, it has been shown that phosphorylation of serine and threonine residues in the C-tail of the receptor by G protein coupled receptor kinases results in uncoupling of the activated receptor from its G proteins, thereby desensitizing the receptor. See Bohm, et al. (1997) J. Biol. Chem. 332:1-18. Further experiments can be performed to determine whether the DC-STAMP protein can be phosphorylated at these serine residues.

Other characteristics of 7 TM proteins include a signature of cysteine residues in the first two extracellular loops, which might form disulphide bridges and stabilize the protein structure. See Savarese and Fraser (1992) J. Biol. Chem. 283:1-19. Also, cysteine residues in the carboxyl tails are potential sites for palmitoylation and may serve to form a fourth intracellular loop. See O'Dowd et al. (1989) J. Biol. Chem. 264:7564-7569; and Strader, et al. (1994) Ann. Rev. Biochem. 63:101-132. The DC-STAMP protein contains an alternative signature of cysteines in TM1 and TM2, and has no cysteine residues in its C-terminus. Combined with the absence of any sequence homology to 7 TM receptors, the described characteristics of the DC-STAMP protein suggest that this novel protein does not belong to any of the existing 7 TM subclasses. DC-STAMP could either form a novel 7 TM protein subclass or be the first member of a new family of multi-membrane spanning proteins.

Characteristic of the DC-STAMP terminus is its very basic amino acid composition. There is some indication that juxtamembrane clusters of positively charged residues in cytoplasmic receptor tails can associate with proteins of the ERM (ezrin, radaxin, moesin) family. See Bretscher (1999) Curr. Op. Cell Biol. 11:109-116. Since these ERM proteins have been implicated as membrane cytoskeletal linkers, this might suggest association of DC-STAMP with the cytoskeleton. Ligation of DC-STAMP might affect adhesive or migratory capacities, essential for proper DC function. These can be tested.

Structural predictions for the type III membrane protein suggest hydrophobic transmembrane segments from about val35-ala5l (TM1); ser57-ser75 (TM2); asn96-ile114 (TM3); tyr144-asp162 (TM4); leu214-phe230 (TM5); leu295-val313 (TM6); and pro379-met398 (TM7). The use of several TM prediction programs for the hydropathy analysis of DC-STAMP resulted in different models, regarding the position and number of transmembrane domains. The data suggest a model in which the DC-STAMP protein contains 7 transmembrane domains. First, the position of potential glycosylation sites, putative phosphorylation recognition sites and the intracellular C-terminus favor this model. Second, based on the presence of charged amino acids, which generally flank transmembrane regions, the model supports a type IIIb integral membrane protein, with the N-terminus of DC-STAMP outside and the C-terminus on the luminal side of the membrane. Finally, the DC-STAMP protein contains a proline residue between TM1 and TM2. Prolines are known to disrupt helices and the proline residue at position 56 in DC-STAMP could help to establish a loop and redirect the protein into the membrane. This could possibly compensate for the rather short hydrophobic stretches of TM1 (transmembrane region 1) and TM2, 17 and 18 amino acids in length, respectively. Also, both TM1 and TM2 contain a pair of cysteine residues, which could further stabilize this intramembrane loop by a disulphide bridge near the outer membrane side.

However, since TM2, TM3, and TM4 are rather weak TM regions, alternative models cannot be excluded comprising 5 or 4 TM regions, in which TM1 and TM2 form a single transmembrane domain and either TM3 or TM4 or both are not present. Only two 5 TM spanning proteins have been described so far, the 865 amino acid AC133 orphan receptor, expressed by hematopoietic stem cells (Miraglia, et al. (1997) Blood 90:5013-5021), and the CD47 molecule (Lindberg, et al. (1993) J. Cell. Biol. 123:485-496). The TM4 superfamily consists of nearly 20 genes, encoding proteins which are thought to be involved in the grouping and stabilization of cell-surface proteins. The DC-STAMP protein however, does not show significant homology to either these TM4 or TM5 proteins, indicating that DC-STAMP represents a novel protein family.

Transmembrane segments are typically 20-25 amino acids in length. Based upon models and data on bacteriorhodopsin, these regions are predicted to be α-helices and be oriented to form a ligand binding pocket. See, e.g., Findley, et al. (1990) Trends Pharmacol. Sci. 11:492-499. Other data suggest that the amino termini of the proteins are extracellular, and the carboxy termini are intracellular. See, e.g., Lodish, et al. (1995) Molecular Cell Biology 3d ed., Scientific American, New York; and Watson and Arkinstall (1994) The G-Protein Linked Receptor FactsBook Academic Press, San Diego, Calif. Phosphorylation cascades have been implicated in the signal transduction pathway of these receptors.

7 TM receptors comprise a family of very heterogeneous proteins that signal through heterotrimeric G proteins (Strader, et al. (1994) Ann. Rev. Biochem 63:101-132), including chemokihe, hormone and photoreceptors. The vast majority of 7 TM proteins are G-protein coupled. The presence of an aspartate in the second transmembrane region and a so-called “DRY or ERY motif”, closely following the third transmembrane region, are both thought to be involved in the signal transduction via G-proteins. See Savarese and Fraser (1992) J. Biol. Chem. 283:1-19; and Bourne (1997) Curr. Op. Cell Biol. 9:134-142. DC-STAMP contains neither of these sequences, but such motifs may possibly not be recognized as such. Similarly, other 7 TM proteins, such as the Duffy antigen receptor (DARC) on erythrocytes and the EGF-7TM receptors, also lack consensus sequences for G protein coupling and no signal transduction via these receptors has been proven as yet. See Horuk, et al. (1996) J. Leukoc. Biol. 59:29-38; and McKnight and Gordon (1996) Immunol. Today 17:283-287.

Although the full spectrum of biological activities mediated by these 7 transmembrane receptors has not been fully determined, chemoattractant effects are recognized. Chemokine receptors are notable members of the GPCR family. See, e.g., Samson, et al. (1996) Biochemistry 35:3362-3367; and Rapport, et al. (1996) J. Leukocyte Biology 59:18-23. The best known biological functions of chemokine molecules relate to chemoattraction of leukocytes. However, new chemokines and receptors are being discovered, and their biological effects on the various cells responsible for immunological responses are topics of continued study.

DC-STAMP agonists, or antagonists, may also act as functional or receptor antagonists, e.g., which block DC interactions or physiology, or mediating the opposite actions. DC are implicated in T cell mediated immunity, which is important in various diseases. T cell immunity is deficient in various contexts, e.g., in tumor immunotherapy and allergic responses. Conversely, it is overactive in autoimmune diseases and transplantation rejection contexts. Thus, DC-STAMP, or its antagonists, may be useful in the treatment of abnormal medical conditions, including immune disorders, e.g., immune deficiencies, chronic inflammation, or tissue rejection, or other physiological conditions. The implication of antigen presentation in initiation of an immune response is a likely condition to be affected by the use of a DC-STAMP related reagent. Compositions combining the DC-STAMP and other DC affecting reagents will often be used. See below.

The DSP-1 forms are highly expressed in mast cells, which are implicated in allergic responses, particularly in release of histamine. See, e.g., Kaliner and Metcalfe (eds. 1992) The Mast Cell in Health and Disease. Reagents related to activation or deactivation of DSP-1 signaling may be important in medical conditions mediated by cells expressing the antigen. Both the long (L) and short (s) forms are type I membrane proteins, and possess cytoplasmic domains with multiple ITIM motifs, suggesting an inhibitory receptor signaling role. See, e.g., Kung, et al. (1999) J. Immunol. 162:5876-87; Carlyle, et al. (1999) J. Immunol. 162:5917-5923; Nakamura, et al. (1997) J. Exp. Med. 185:673-684; Olcese, et al. (1996) J. Immunol. 156:4531-4534; and Daeron, et al. (1995) Immunity 3:635-646. The transmembrane segments correspond approximately to residues 172-188 (166-198) for the L form, and 59-75 (53-85) of the S form. However the actual boundaries of transmembrane segments may vary or depend upon kinetic and other factors.

These natural antigens will be capable of mediating various biochemical responses which lead to biological or physiological responses in target cells. The preferred embodiments characterized herein are from primate, e.g., human, but other species counterparts will exist in nature. Additional sequences for proteins in other mammalian species, e.g., primates, canines, felines, and rodents, should also be available, particularly the domestic animal species. See below. The descriptions below are directed, for exemplary purposes, to a human DC-STAMP or DSP-1, but are likewise applicable to related embodiments from other species.

II. Purified DC-STAMP or DSP-1

Primate, e.g., human, DC-STAMP or DSP-1 amino acid sequences, are shown in Tables 1 or 2. Other naturally occurring nucleic acids which encode the proteins can be isolated by standard procedures using the provided sequences, e.g., PCR techniques, or by hybridization. Primer extension or RACE methods can extend to adjacent sequence, either on message or genomic. These amino acid sequences, provided amino to carboxy, are important in providing sequence information for the proteins allowing for distinguishing the protein antigen from other proteins and exemplifying numerous variants. Moreover, the peptide sequences allow preparation of peptides to generate antibodies to recognize such segments, and nucleotide sequences allow preparation of oligonucleotide probes, both of which are strategies for detection or isolation, e.g., cloning, of genes encoding such sequences.

As used herein, the term “human DC-STAMP” shall encompass, when used in a protein context, a protein having amino acid sequence corresponding to a polypeptide shown in SEQ ID NO: 2, or significant fragments thereof. Preferred embodiments comprise a plurality of distinct, e.g., nonoverlapping, segments of the specified length. Typically, the plurality will be at least two, more usually at least three, and preferably 5, 7, or even more. While the length minima are provided, longer lengths, of various sizes, may be appropriate, e.g., one of length 7, and two of length 12. Similarly with the term DSP-1 and SEQ ID NO: 4 and 6.

Binding components, e.g., antibodies, typically bind to an antigen with high affinity, e.g., at least about 100 nM, usually better than about 30 nM, preferably better than about 10 nM, and more preferably at better than about 3 nM. Counterpart proteins will be found in mammalian species other than human, e.g., other primates, ungulates, or rodents. Non-mammalian species should also possess structurally or functionally related genes and proteins, e.g., birds or fish.

The term “polypeptide” as used herein includes a significant fragment or segment, and encompasses a stretch of amino acid residues of at least about 8 amino acids, generally at least about 12 amino acids, typically at least about 16 amino acids, preferably at least about 20 amino acids, and, in particularly preferred embodiments, at least about 30 or more amino acids, e.g., 35, 40, 45, 50, etc. Such fragments may have ends which begin and/or end at virtually all positions, e.g., beginning at residues 1, 2, 3, etc., and ending at, e.g., 150, 149, 148, etc., in all practical combinations. Particularly interesting peptides have ends corresponding to structural domain boundaries, e.g., transmembrane segments or identified motifs. See Tables 1 and 2.

The term “binding composition” refers to molecules that bind with specificity to DC-STAMP or DSP-1, e.g., in an antibody-antigen interaction. The specificity may be more or less inclusive, e.g., specific to a particular embodiment, or to groups of related embodiments, e.g., primate, rodent, etc. It also includes compounds, e.g., proteins, which specifically associate with DC-STAMP, including in a natural physiologically relevant protein-protein interaction, either covalent or non-covalent. The molecule may be a polymer, or chemical reagent. A functional analog may be a protein with structural modifications, or it may be a molecule which has a molecular shape which interacts with the appropriate binding determinants. The compounds may serve as agonists or antagonists of a receptor binding interaction, see, e.g., Goodman, et al. (eds.) Goodman & Gilman's: The Pharmacological Bases of Therapeutics (current ed.) Pergamon Press.

Substantially pure, e.g., in a protein context, typically means that the protein is free from other contaminating proteins, nucleic acids, or other biologicals derived from the original source organism. Purity may be assayed by standard methods, typically by weight, and will ordinarily be at least about 40% pure, generally at least about 50% pure, often at least about 60% pure, typically at least about 80% pure, preferably at least about 90% pure, and in most preferred embodiments, at least about 95% pure. Carriers or excipients will often be added.

Solubility of a polypeptide or fragment depends upon the environment and the polypeptide. Many parameters affect polypeptide solubility, including temperature, electrolyte environment, size and molecular characteristics of the polypeptide, and nature of the solvent. Typically, the temperature at which the polypeptide is used ranges from about 4° C. to about 65° C. Usually the temperature at use is greater than about 18° C. For diagnostic purposes, the temperature will usually be about room temperature or warmer, but less than the denaturation temperature of components in the assay. For therapeutic purposes, the temperature will usually be body temperature, typically about 37° C. for humans and mice, though under certain situations the temperature may be raised or lowered in situ or in vitro.

The size and structure of the polypeptide should generally be in a substantially stable state, and usually hot in a denatured state. The polypeptide may be associated with other polypeptides in a quaternary structure, e.g., to confer solubility, or associated with lipids or detergents.

The solvent and electrolytes will usually be a biologically compatible buffer, of a type used for preservation of biological activities, and will usually approximate a physiological aqueous solvent. Usually the solvent will have a neutral pH, typically between about 5 and 10, and preferably about 7.5. On some occasions, one or more detergents will be added, typically a mild non-denaturing one, e.g., CHS (cholesteryl hemisuccinate) or CHAPS (3-[3-cholamidopropyl)dimethylammonio]-1-propane sulfonate), or a low enough concentration as to avoid significant disruption of structural or physiological properties of the protein. In other instances, a harsh detergent may be used to effect significant denaturation.

III. Physical Variants

This invention also encompasses proteins or peptides having substantial amino acid sequence identity with the amino acid sequences of the DC-STAMP or DSP-1 antigens. The variants include species, polymorphic, or allelic variants.

Amino acid sequence homology, or sequence identity, is determined by optimizing residue matches, if necessary, by introducing gaps as required. See also Needleham, et al. (1970) J. Mol. Biol. 48:443-453; Sankoff, et al. (1983) Chapter One in Time Warps, String Edits, and Macromolecules: The Theory and Practice of Sequence Comparison, Addison-Wesley, Reading, Mass.; and software packages from IntelliGenetics, Mountain View, Calif.; and the University of Wisconsin Genetics Computer Group, Madison, Wis. Sequence identity changes when considering conservative substitutions as matches. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine. The conservation may apply to biological features, functional features, or structural features. Homologous amino acid sequences are typically intended to include natural polymorphic or allelic and interspecies variations of a protein sequence. Typical homologous proteins or peptides will have from 25-100% identity (if gaps can be introduced), to 50-100% identity (if conservative substitutions are included) with the amino acid sequences of the antigens. Identity measures will be at least about 35%, generally at least about 40%, often at least about 50%, typically at least about 60%, usually at least about 70%, preferably at least about 80%, and more preferably at least about 90%.

The isolated DC-STAMP or DSP-1 DNA can be readily modified by nucleotide substitutions, nucleotide deletions, nucleotide insertions, and inversions of short nucleotide stretches. These modifications may result in novel DNA sequences which encode these antigens, their derivatives, or proteins having similar physiological, immunogenic, antigenic, or other functional activity. These modified sequences can be used to produce mutant antigens or to enhance expression. Enhanced expression may involve gene amplification, increased transcription, increased translation, and other mechanisms. “Mutant DC-STAMP” encompasses a polypeptide otherwise falling within the sequence identity definition of the DC-STAMP as set forth above, but having an amino acid sequence which differs from that of the antigen as normally found in nature, whether by way of deletion, substitution, or insertion. This generally includes proteins having significant identity with a protein having sequence of SEQ ID NO: 2, and as sharing various biological activities, e.g., antigenic or immunogenic, with those sequences, and in preferred embodiments contain most of the natural full length disclosed sequences. Full length sequences will typically be preferred, though truncated versions will also be useful, likewise, genes or proteins found from natural sources are typically most desired. Similar concepts apply to different DC-STAMP proteins, particularly those found in various warm blooded animals, e.g., mammals and birds. These descriptions are generally meant to encompass many DC-STAMP proteins, not limited to the particular primate embodiments specifically discussed.

DC-STAMP or DSP-1 mutagenesis can also be conducted by making amino acid insertions or deletions. Substitutions, deletions, insertions, or any combinations may be generated to arrive at a final construct. Insertions include amino- or carboxy-terminal fusions. Random mutagenesis can be conducted at a target codon and the expressed mutants can then be screened for the desired activity. Methods for making substitution mutations at predetermined sites in DNA having a known sequence are well known in the art, e.g., by M13 primer mutagenesis or polymerase chain reaction (PCR) techniques. See, e.g., Sambrook, et al. (1989); Ausubel, et al. (1987 and Supplements); and Kunkel, et al. (1987) Methods in Enzymol. 154:367-382. Preferred embodiments include, e.g., 1-fold, 2-fold, 3-fold, 5-fold, 7-fold, etc., preferably conservative substitutions at the nucleotide or amino acid levels. Preferably the substitutions will be away from the conserved cysteines, and often will be in the regions away from the extramembrane domains. Such variants may be useful to produce specific antibodies, and often will share many or all biological properties.

The present invention also provides recombinant proteins, e.g., heterologous fusion proteins using segments from these proteins. A heterologous fusion protein is a fusion of proteins or segments which are naturally not normally fused in the same manner. A similar concept applies to heterologous nucleic acid sequences.

In addition, new constructs may be made from combining similar functional domains from other proteins. For example, target-binding or other segments may be “swapped” between different new fusion polypeptides or fragments. See, e.g., Cunningham, et al. (1989) Science 243:1330-1336, and O'Dowd, et al. (1988) J. Biol. Chem. 263:15985-15992.

The phosphoramidite method described by Beaucage and Carruthers (1981) Tetra. Letts. 22:1859-1862, will produce useful synthetic DNA fragments. A double stranded fragment will often be obtained either by synthesizing the complementary-strand and annealing the strand together under appropriate conditions or by adding the complementary strand using DNA polymerase with an appropriate primer sequence, e.g., PCR techniques.

Structural analysis can be applied to this gene, in comparison to members of related gene families, e.g., GPCRs. In particular, β-sheet and α-helix residues can be determined using, e.g., RASMOL program, see Bazan, et al. (1996) Nature 379:591; Lodi, et al. (1994) Science 263:1762-1766; Sayle and Milner-White (1995) TIBS 20:374-376; and Gronenberg, et al. (1991) Protein Engineering 4:263-269. Preferred residues for substitutions include the surface exposed extramembrane residues which would be predicted to interact with a counterstructure or ligand. Other residues which should conserve function will be conservative substitutions, particularly at a position far from the surface exposed residues, e.g., an intramembrane residue.

IV. Functional Variants

The blocking of physiological response to DC-STAMP or DSP-1 may result from the competitive inhibition of binding of a ligand or counterstructure to the antigen.

In vitro assays of the present invention will often use isolated protein, soluble fragments comprising receptor binding segments of these proteins, or fragments attached to solid phase substrates. These assays will also allow for the diagnostic determination of the effects of either binding segment mutations and modifications, or ligand mutations and modifications.

This invention also contemplates the use of competitive drug screening assays, e.g., where neutralizing antibodies to the antigen, or receptor binding fragments compete with a test compound.

“Derivatives” of DC-STAMP or DSP-1 antigens include amino acid sequence mutants from naturally occurring forms, glycosylation variants, and covalent or aggregate conjugates with other chemical moieties. Covalent derivatives can be prepared by linkage of functionalities to groups which are found in amino acid side chains or at the N- or C-termini, e.g., by standard means. See, e.g., Lundblad and Noyes (1988) Chemical Reagents for Protein Modification, vols. 1-2, CRC Press, Inc., Boca Raton, Fla.; Hugli (ed. 1989) Techniques in Protein Chemistry, Academic Press, San Diego, Calif.; and Wong (1991) Chemistry of Protein Conjugation and Cross Linking, CRC Press, Boca Raton, Fla.

In particular, glycosylation alterations are included, e.g., made by modifying the glycosylation patterns of a polypeptide during its synthesis and processing, or in further processing steps. See, e.g., Elbein (1987) Ann. Rev. Biochem. 56:497-534. Also embraced are versions of the peptides with the same primary amino acid sequence which have other minor modifications, including phosphorylated amino acid residues, e.g., phosphotyrosine, phosphoserine, or phosphothreonine.

Fusion polypeptides between DC-STAMP or DSP-1 and other homologous or heterologous proteins are also provided. Many 7TM receptors or other surface proteins are multimeric, e.g., homodimeric entities, and a repeat construct may have various advantages, including lessened susceptibility to proteolytic cleavage. Typical examples are fusions of a reporter polypeptide, e.g., luciferase, with a segment or domain of a protein, e.g., a receptor-binding segment, so that the presence or location of the fused ligand may be easily determined. See, e.g., Dull, et al., U.S. Pat. No. 4,859,609. Other gene fusion partners include bacterial β-galactosidase, trpE, Protein A, β-lactamase, alpha amylase, alcohol dehydrogenase, yeast alpha mating factor, and detection or purification tags such as a FLAG sequence of His6 sequence. See, e.g., Godowski, et al. (1988) Science 241:812-816.

Fusion peptides will typically be made by either recombinant nucleic acid methods or by synthetic polypeptide methods. Techniques for nucleic acid manipulation and expression are described generally, e.g., in Sambrook, et al. (1989) Molecular Cloning: A Laboratory Manual (2d ed.), vols. 1-3, Cold Spring Harbor Laboratory; and Ausubel, et al. (eds. 1993) Current Protocols in Molecular Biology, Greene and Wiley, NY. Techniques for synthesis of polypeptides are described, e.g., in Merrifield (1963) J. Amer. Chem. Soc. 85:2149-2156; Merrifield (1986) Science 232: 341-347; Atherton, et al. (1989) Solid Phase Peptide Synthesis: A Practical Approach, IRL Press, Oxford; and Grant (1992) Synthetic Peptides: A User's Guide, W.H. Freeman, NY. Refolding methods, e.g., with membranes, may be applicable to synthetic proteins.

This invention also contemplates the use of derivatives of DC-STAMP or DSP-1 proteins other than variations in amino acid sequence or glycosylation. Such derivatives may involve covalent or aggregative association with chemical moieties or protein carriers. Covalent or aggregative derivatives will be useful as immunogens, as reagents in immunoassays, or in purification methods such as for affinity purification of binding partners, e.g., other antigens. A DC-STAMP or DSP-1 can be immobilized by covalent bonding to a solid support such as cyanogen bromide-activated SEPHAROSE, by methods which are well known in the art, or adsorbed onto polyolefin surfaces, with or without glutaraldehyde cross-linking, for use in the assay or purification of antibodies or an alternative binding composition. These proteins can also be labeled with a detectable group, e.g., for use in diagnostic assays. Purification of antigen may be effected by an immobilized antibody or complementary binding partner, e.g., binding portion of a receptor.

A solubilized fragment of this invention can be used as an immunogen for the production of antisera or antibodies specific for binding. Purified antigen can be used to screen monoclonal antibodies or antigen-binding fragments, encompassing antigen binding fragments of natural antibodies, e.g., Fab, Fab′, F(ab)₂, etc. Purified DC-STAMP or DSP-1 antigens can also be used as a reagent to detect antibodies generated in response to the presence of elevated levels of the antigen, which may be diagnostic of an abnormal or specific physiological or disease condition. This invention contemplates antibodies raised against amino acid sequences encoded by nucleotide sequence shown in SEQ ID NO: 1 or 4 or 6, or fragments of proteins containing it. In particular, this invention contemplates antibodies having binding affinity to or being raised against specific domains, e.g., extracellular segments.

The present invention contemplates the isolation of additional closely related species variants. Southern and Northern blot analysis will establish that similar genetic entities exist in other mammals. It is likely that antigens are widespread in species variants, e.g., rodents, lagomorphs, carnivores, artiodactyla, perissodactyla, and primates.

The invention also provides means to isolate a group of related antigens displaying both distinctness and similarities in structure, expression, and function. Elucidation of many of the physiological effects of the molecules will be greatly accelerated by the isolation and characterization of additional distinct species or polymorphic variants of them. In particular, the present invention provides useful probes for identifying additional homologous genetic entities in different species.

The isolated genes will allow transformation of cells lacking expression of DC-STAMP or DSP-1, e.g., either species types or cells which lack corresponding proteins and exhibit negative background activity. This should allow analysis of the function of antigen in comparison to untransformed control cells.

Dissection of critical structural elements which effect the various physiological functions mediated through these antigens is possible using standard techniques of modern molecular biology, particularly in comparing members of the related class. See, e.g., the homolog-scanning mutagenesis technique described in Cunningham, et al. (1989) Science 243:1339-1336; and approaches used in O'Dowd, et al. (1988) J. Biol. Chem. 263:15985-15992; and Lechleiter, et al. (1990) EMBO J. 9:4381-4390.

Intracellular functions would probably involve receptor signaling. However, protein internalization may occur under certain circumstances, and interaction between intracellular components and ligand or receptor may occur. Specific segments of interaction of membrane antigen with interacting components may be identified by mutagenesis or direct biochemical means, e.g., cross-linking or affinity methods. Structural analysis by crystallographic or other physical methods will also be applicable. Further investigation of the mechanism of signal transduction will include study of associated components which may be isolatable by affinity methods or by genetic means, e.g., complementation analysis of mutants.

Further study of the expression and control of DC-STAMP or DSP-1 will be pursued. The controlling elements associated with the antigens should exhibit differential physiological, developmental, tissue specific, or other expression patterns. Upstream or downstream genetic regions, e.g., control elements, are of interest.

Structural studies of the membrane antigens will lead to design of new antigens, particularly analogs exhibiting agonist or antagonist properties on the molecule. This can be combined with previously described screening methods to isolate antigens exhibiting desired spectra of activities.

V. Antibodies

Antibodies can be raised to various epitopes of the membrane proteins, including species, polymorphic, or allelic variants, and fragments thereof, both in their naturally occurring forms and in their recombinant forms. Additionally, antibodies can be raised to the proteins in either their active forms or in their inactive forms, including native or denatured versions. Anti-idiotypic antibodies are also contemplated.

Antibodies, including binding fragments and single chain versions, against predetermined fragments of the antigens can be raised by immunization of animals with conjugates of the fragments with immunogenic proteins. Monoclonal antibodies are prepared from cells secreting the desired antibody. These antibodies can be screened for binding to normal or defective proteins, or screened for agonistic or antagonistic activity, e.g., mediated through a receptor. Antibodies may be agonistic or antagonistic, e.g., by sterically blocking binding to a receptor. These monoclonal antibodies will usually bind with at least a KD of about 1 mM, more usually at least about 300 μM, typically at least about 100 μM, more typically at least about 30 μM, preferably at least about 10 μM, and more preferably at least about 3 μM or better.

A DC-STAMP or DSP-1 protein that specifically binds to or that is specifically immunoreactive with an antibody generated against a defined immunogen, such as an immunogen consisting of the amino acid sequence of SEQ ID,NO: 2, is typically determined in an immunoassay. The immunoassay typically uses a polyclonal antiserum which was raised, e.g., to a protein of SEQ ID NO: 2. This antiserum is selected to have low crossreactivity against other related proteins, e.g., human or rodent DC-STAMP, preferably from the same species, and any such crossreactivity is removed by immunoabsorption prior to use in the immunoassay.

In order to produce antisera for use in an immunoassay, the protein of SEQ ID NO: 2, or a combination thereof, is isolated as described herein. For example, recombinant protein may be produced in a mammalian cell line. An appropriate host, e.g., an inbred strain of mice such as Balb/c, is immunized with the selected protein, typically using a standard adjuvant, such as Freund's adjuvant, and a standard mouse immunization protocol (see Harlow and Lane, supra). Alternatively, a synthetic peptide derived from the sequences disclosed herein and conjugated to a carrier protein can be used an immunogen. Polyclonal sera are collected and titered against the immunogen protein in an immunoassay, e.g., a solid phase immunoassay with the immunogen immobilized on a solid support. Polyclonal antisera with a titer of 10⁴ or greater are selected and tested for their cross reactivity against other related family members, e.g., rodent DC-STAMP, using a competitive binding immunoassay such as the one described in Harlow and Lane, supra, at pages 570-573. Preferably at least one other related family member is used in this determination in conjunction with, e.g., the primate embodiment. The desired target family members can be produced as recombinant proteins and isolated using standard molecular biology and protein chemistry techniques as described herein.

Immunoassays in the competitive binding format can be used for the crossreactivity determinations. For example, the protein of SEQ ID NO: 2 can be immobilized to a solid support. Proteins added to the assay compete with the binding of the antisera to the immobilized antigen. The ability of the above proteins to compete with the binding of the antisera to the immobilized protein is compared to the protein of SEQ ID NO: 2. The percent crossreactivity for the above proteins is calculated, using standard calculations. Those antisera with less than 10% crossreactivity with each of the proteins listed above are selected and pooled. The cross-reacting antibodies are then removed from the pooled antisera by immunoabsorption with the above-listed proteins.

The immunoabsorbed and pooled antisera are then used in a competitive binding immunoassay as described above to compare a second protein to the immunogen protein (e.g., the protein of SEQ ID NO: 2). In order to make this comparison, the two proteins are each assayed at a wide range of concentrations and the amount of each protein required to inhibit 50% of the binding of the antisera to the immobilized protein is determined. If the amount of the second protein required is less than twice the amount of the protein of the selected protein or proteins that is required, then the second protein is said to specifically bind to an antibody generated to the immunogen.

The antibodies of this invention can also be useful in diagnostic applications. As capture or non-neutralizing antibodies, they can be screened for ability to bind to the antigens without inhibiting binding to a receptor. As neutralizing antibodies, they can be useful in competitive binding assays. They will also be useful in detecting or quantifying DC-STAMP or DSP-1 protein or their receptors. See, e.g., Chan (ed. 1987) Immunology: A Practical Guide, Academic Press, Orlando, Fla.; Price and Newman (eds. 1991) Principles and Practice of Immunoassay, Stockton Press, N.Y.; and Ngo (ed. 1988) Nonisotopic Immunoassay, Plenum Press, N.Y. Cross absorptions, depletions, or other means will provide. preparations of defined selectivity, e.g., unique or shared species specificities. These may be the basis for tests which will identify various groups of antigens.

Further, the antibodies, including antigen binding fragments, of this invention can be potent antagonists that bind to the antigen and inhibit functional binding, e.g., to a receptor which may elicit a biological response. They also can be useful as non-neutralizing antibodies and can be coupled to toxins or radionuclides so that when the antibody binds to antigen, a cell expressing it, e.g., on its surface, is killed. Further, these antibodies can be conjugated to drugs or other therapeutic agents, either directly or indirectly by means of a linker, and may effect drug targeting.

Antigen fragments may be joined to other materials, particularly polypeptides, as fused or covalently joined polypeptides to be used as immunogens. An antigen and its fragments may be fused or covalently linked to a variety of immunogens, such as keyhole limpet hemocyanin, bovine serum albumin, tetanus toxoid, etc. See Microbiology, Hoeber Medical Division, Harper and Row, 1969; Landsteiner (1962) Specificity of Serological Reactions, Dover Publications, New York; Williams, et al. (1967) Methods in Immunology and Immunochemistry, vol. 1, Academic Press, New York; and Harlow and Lane (1988) Antibodies: A Laboratory Manual, CSH Press, NY, for descriptions of methods of preparing polyclonal antisera.

In some instances, it is desirable to prepare monoclonal antibodies from various mammalian hosts, such as mice, rodents, primates, humans, etc. Description of techniques for preparing such monoclonal antibodies may be found in, e.g., Stites, et al. (eds.) Basic and Clinical Immunology (4th ed.), Lange Medical Publications, Los Altos, Calif., and references cited therein; Harlow and Lane (1988) Antibodies: A Laboratory Manual, CSH Press; Goding (1986) Monoclonal Antibodies: Principles and Practice (2d ed.), Academic Press, New York; and particularly in Kohler and Milstein (1975) in Nature 256:495-497, which discusses one method of generating monoclonal antibodies.

Other suitable techniques involve in vitro exposure of lymphocytes to the antigenic polypeptides or alternatively to selection of libraries of antibodies in phage or similar vectors. See, Huse, et al. (1989) “Generation of a Large Combinatorial Library of the Immunoglobulin Repertoire in Phage Lambda,” Science 246:1275-1281; and Ward, et al. (1989) Nature 341:544-546. The polypeptides and antibodies of the present invention may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents, teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see Cabilly, U.S. Pat. No. 4,816,567; Moore, et al., U.S. Pat. No. 4,642,334; and Queen, et al.. (1989) Proc. Nat'l Acad. Sci. USA 86:10029-10033.

The antibodies of this invention can also be used for affinity chromatography in isolating the protein. Columns can be prepared where the antibodies are linked to a solid support. See, e.g., Wilchek et al. (1984) Meth. Enzymol. 104:3-55. The converse may be used to purify antibodies.

Antibodies raised against DC-STAMP or DSP-1 will also be useful to raise anti-idiotypic antibodies. These will be useful in detecting or diagnosing various immunological conditions related to expression of the respective antigens.

VI. Nucleic Acids

The described peptide sequences and the related reagents are useful in detecting, isolating, or identifying a DNA clone encoding DC-STAMP or DSP-1, e.g., from a natural source. Typically, it will be useful in isolating a gene from mammal, and similar procedures will be applied to isolate genes from other species, e.g., warm blooded animals, such as birds and mammals. Cross hybridization will allow isolation of DC-STAMP from the same, e.g., polymorphic variants, or other species. A number of different approaches will be available to successfully isolate a suitable nucleic acid clone.

The purified protein or defined peptides are useful for generating antibodies by standard methods, as described above. Synthetic peptides or purified protein can be presented to an immune system to generate monoclonal or polyclonal antibodies. See, e.g., Coligan (1991) Current Protocols in Immunology Wiley/Greene; and Harlow and Lane (1989) Antibodies: A Laboratory Manual, Cold Spring Harbor Press.

For example, the specific binding composition could be used for screening of an expression library made from a cell line which expresses a DC-STAMP. Screening of intracellular expression can be performed by various staining or immunofluorescence procedures. Binding compositions could be used to affinity purify or sort out cells expressing a surface fusion protein.

The peptide segments can also be used to predict appropriate oligonucleotides to screen a library. The genetic code can be used to select appropriate oligonucleotides useful as probes for screening. See, e.g., SEQ ID NO: 1 or 4 or 6. In combination with polymerase chain reaction (PCR) techniques, synthetic oligonucleotides will be useful in selecting correct clones from a library. Complementary sequences will also be used as probes, primers, or antisense strands. Various fragments should be particularly useful, e.g., coupled with anchored vector or poly-A complementary PCR techniques or with complementary DNA of other peptides.

This invention contemplates use of isolated DNA or fragments to encode an antigenic or biologically active corresponding polypeptide, particularly lacking the portion coding an untranslated 5′ portion of the described sequence. In addition, this invention covers isolated or recombinant DNA which encodes a biologically active protein or polypeptide and which is capable of hybridizing under appropriate conditions with the DNA sequences described herein. Said biologically active protein or polypeptide can be an intact antigen, or fragment, and have an amino acid sequence disclosed in, e.g., SEQ ID NO: 2 or 5 or 7, particularly a mature, secreted polypeptide. Further, this invention covers the use of isolated or recombinant DNA, or fragments thereof, which encode proteins which exhibit high identity to membrane DC-STAMP or DSP-1. The isolated DNA can have the respective regulatory sequences in the 5′ and 3′ flanks, e.g., promoters, enhancers, poly-A addition signals, and others. Alternatively, expression may be effected by operably linking a coding segment to a heterologous promoter, e.g., by inserting a promoter upstream from an endogenous gene.

An “isolated” nucleic acid is a nucleic acid, e.g., an RNA, DNA, or a mixed polymer, which is substantially separated from other components which naturally accompany a native sequence, e.g., ribosomes, polymerases, and/or flanking genomic sequences from the originating species. The term embraces a nucleic acid sequence which has been removed from its naturally occurring environment, and includes recombinant or cloned DNA isolates and chemically synthesized analogs or analogs biologically synthesized by heterologous systems. A substantially pure molecule includes isolated forms of the molecule. Generally, the nucleic acid will be in a vector or fragment less than about 50 kb, usually less than about 30 kb, typically less than about 10 kb, and preferably less than about 6 kb.

An isolated nucleic acid will generally be a homogeneous composition of molecules, but will, in some embodiments, contain minor heterogeneity. This heterogeneity is typically found at the polymer ends or portions not critical to a desired biological function or activity.

A “recombinant” nucleic acid is defined either by its method of production or its structure. In reference to its method of production, e.g., a product made by a process, the process is use of recombinant nucleic acid techniques, e.g., involving human intervention in the nucleotide sequence, typically selection or production. Alternatively, it can be a nucleic acid made by generating a sequence comprising fusion of two fragments which are not naturally contiguous to each other, but is meant to exclude products of nature, e.g., naturally occurring mutants. Thus, e.g., products made by transforming cells with a nonnaturally occurring vector is encompassed, as are nucleic acids comprising sequence derived using any synthetic oligonucleotide process. Such is often done to replace a codon with a redundant codon encoding the same or a conservative amino acid, while typically introducing or removing a sequence recognition site.

Alternatively, it is performed to join together nucleic acid segments of desired functions to generate a single genetic entity comprising a desired combination of functions not found in the commonly available natural forms. Restriction enzyme recognition sites are often the target of such artificial manipulations, but other site specific targets, e.g., promoters, DNA replication sites, regulation sequences, control sequences, or other useful features may be incorporated by design. A similar concept is intended for a recombinant, e.g., fusion, polypeptide. Specifically included are synthetic nucleic acids which, by genetic code redundancy, encode polypeptides similar to fragments of these antigens, and fusions of sequences from various different species or polymorphic variants.

A significant “fragment” in a nucleic acid context is a contiguous segment of at least about 17 nucleotides, generally at least about 22 nucleotides, ordinarily at least about 29 nucleotides, more often at least about 35 nucleotides, typically at least about 41 nucleotides, usually at least about 47 nucleotides, preferably at least about 55 nucleotides, and in particularly preferred embodiments will be at least about 60 or more nucleotides, e.g., 67, 73, 81, 89, 95, etc.

A DNA which codes for a DC-STAMP or DSP-1 protein will be particularly useful to identify genes, mRNA, and cDNA species which code for related or similar proteins, as well as DNAs which code for homologous proteins from different species. There will be homologs in other species, including primates, rodents, canines, felines, birds, and fish. Various DC-STAMP or DSP-1 proteins should be homologous and are encompassed herein. However, even proteins that have a more distant evolutionary relationship to the antigen can readily be isolated under appropriate conditions using these sequences if they are sufficiently homologous. Primate membrane proteins are of particular interest.

Recombinant clones derived from the genomic sequences, e.g., containing introns, will be useful for transgenic studies, including, e.g., transgenic cells and organisms, and for gene therapy. See, e.g., Goodnow (1992) “Transgenic Animals” in Roitt (ed.) Encyclopedia of Immunology, Academic Press, San Diego, pp. 1502-1504; Travis (1992) Science 256:1392-1394; Kuhn, et al. (1991) Science 254:707-710; Capecchi (1989) Science 244:1288; Robertson (ed. 1987) Teratocarcinomas and Embryonic Stem Cells: A Practical Approach, IRL Press, Oxford; Rosenberg (1992) J. Clinical Oncology 10:180-199; and Cournoyer and Caskey (1993) Ann. Rev. Immunol. 11:297-329. Alternatively, expression may be effected by operably linking a coding segment to a heterologous promoter, e.g., by inserting a promoter upstream from an endogenous gene. See, e.g., Treco, et al. WO96/29411 or U.S. Ser. No. 08/406,030.

Substantial homology, e.g., identity, in the nucleic acid sequence comparison context means either that the segments, or their complementary strands, when compared, are identical when optimally aligned, with appropriate nucleotide insertions or deletions, in at least about 50% of the nucleotides, generally at least about 58%, ordinarily at least about 65%, often at least about 71%, typically at least about 77%, usually at least about 85%, preferably at least about 95 to 98% or more, and in particular embodiments, as high as about 99% or more of the nucleotides. Alternatively, substantial homology exists when the segments will hybridize under selective hybridization conditions, to a strand, or its complement, typically using a sequence of DC-STAMP or DSP-1, e.g., in SEQ ID NO: 1 or 4 or 6. Typically, selective hybridization will occur when there is at least about 55% identity over a stretch of at least about 30 nucleotides, preferably at least about 75% over a stretch of about 25 nucleotides, and most preferably at least about 90% over about 20 nucleotides. See, Kanehisa (1984) Nuc. Acids Res. 12:203-213. The length of identity comparison, as described, may be over longer stretches, and in certain embodiments will be over a stretch of at least about 17 nucleotides, usually at least about 28 nucleotides, typically at least about 40 nucleotides, and preferably at least about 75 to 100 or more nucleotides.

Stringent conditions, in referring to homology in the hybridization context, will be stringent combined conditions of salt, temperature, organic solvents, and other parameters, typically those controlled in hybridization reactions. Stringent temperature conditions will usually include temperatures in excess of about 30° C., usually in excess of about 37° C., typically in excess of about 55° C., 60° C., or 65° C., and preferably in excess of about 70° C. Stringent salt conditions will ordinarily be less than about 1000 mM, usually less than about 400 mM, typically less than about 250 mM, preferably less than about 150 mM, including about 100, 50, or even 20 mM. However, the combination of parameters is much more important than the measure of any single parameter. See, e.g., Wetmur and Davidson (1968) J. Mol. Biol. 31:349-370. Hybridization under stringent conditions should give a background of at least 2-fold over background, preferably at least 3-5 or more.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optical alignment of sequences for comparison can be conducted, e.g., by the local homology algorithm of Smith and Waterman (1981) Adv. Apol. Math. 2:482, by the homology alignment algorithm of Needleman and Wunsch (1970) J. Mol. Biol. 48:443, by the search for similarity method of Pearson and Lipman (1988) Proc. Nat'l Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendrogram showing the clustering relationships used to create the alignment. PILEUP uses a simplification of the progressive alignment method of Feng and Doolittle (1987) J. Mol. Evol. 35:351-360. The method used is similar to the method described by Higgins and Sharp (1989) CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described Altschul, et al. (1990) J. Mol. Biol. 215:403-410. Software for performing BLAST analyses is publicly available through the National Center for Biotechnology Information (http:www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul, et al., supra). These initial neighborhood word hits act as seeds for initiating searches to find longer HSPs containing them. The word hits are then extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff and Henikoff (1989) Proc. Nat'l Acad. Sci. USA 89:10915) alignments (B) of 50, expectation (E) of 10, M=5, N=4, and a comparison of both strands.

In addition to calculating percent sequence identity, the BLAST algorithm also performs a statistical analysis of the similarity between two sequences (see, e.g., Karlin and Altschul (1993) Proc. Nat'l Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences of polypeptides are substantially identical is that the polypeptide encoded by the first nucleic acid is. immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

DC-STAMP or DSP-1 from other mammalian species can be cloned and isolated by cross-species hybridization of closely related species. Homology may be relatively low between distantly related species, and thus hybridization of relatively closely related species is advisable. Alternatively, preparation of an antibody preparation which exhibits less species specificity may be useful in expression cloning approaches.

VII. Making DC-STAMP or DSP-1; Mimetics

DNA which encodes the DC-STAMP or DSP-1 or fragments thereof can be obtained by chemical synthesis, screening cDNA libraries, or screening genomic libraries prepared from a wide variety of cell lines or tissue samples. See, e.g., Okayama and Berg (1982) Mol. Cell. Biol. 2:161-170; Gubler and Hoffman (1983) Gene 25:263-269; and Glover (ed. 1984) DNA Cloning: A Practical Approach, IRL Press, Oxford. Alternatively, the sequences provided herein provide useful PCR primers or allow synthetic or other preparation of suitable genes encoding a DC-STAMP or DSP-1; including naturally occurring embodiments.

This DNA can be expressed in a wide variety of host cells for the synthesis of a full-length DC-STAMP or DSP-1 or fragments which can in turn, e.g., be used to generate polyclonal or monoclonal antibodies; for binding studies; for construction and expression of modified molecules; and for structure/function studies.

Vectors, as used herein, comprise plasmids, viruses, bacteriophage, integratable DNA fragments, and other vehicles which enable the integration of DNA fragments into the genome of the host. See, e.g., Pouwels, et al. (1985 and Supplements) Cloning Vectors: A Laboratory Manual, Elsevier, N.Y.; and Rodriguez, et al. (eds. 1988) Vectors: A Survey of Molecular Cloning Vectors and Their Uses, Buttersworth, Boston, Mass.

For purposes of this invention, DNA sequences are operably linked when they are functionally related to each other. For example, DNA for a presequence or secretory leader is operably linked to a polypeptide if it is expressed as a preprotein or participates in directing the polypeptide to the cell membrane or in secretion of the polypeptide. A promoter is operably linked to a coding sequence if it controls the transcription of the polypeptide; a ribosome binding site is operably linked to a coding sequence if it is positioned to permit translation. Usually, operably linked means contiguous and in reading frame, however, certain genetic elements such as repressor genes are not contiguously linked but still bind to operator sequences that in turn control expression. See, e.g., Rodriguez, et al., Chapter 10, pp. 205-236; Balbas and Bolivar (1990) Methods in Enzymoloay 185:14-37; and Ausubel, et al. (1993) Current Protocols in Molecular Biology, Greene and Wiley, NY.

Representative examples of suitable expression vectors include pCDNA1; pCD, see Okayama, et al. (1985) Mol. Cell Biol. 5:1136-1142; pMClneo Poly-A, see Thomas, et al. (1987) Cell 51:503-512; and a baculovirus vector such as pAC 373 or pAC 610. See, e.g., Miller (1988) Ann. Rev. Microbiol. 42:177-199.

It will often be desired to express a DC-STAMP or DSP-1 polypeptide in a system which provides a specific or defined glycosylation pattern. See, e.g., Luckow and Summers (1988) Bio/Technology 6:47-55; and Kaufman (1990) Meth. Enzymol. 185:487-511.

The DC-STAMP or DSP-1, or a fragment thereof, may be engineered to be phosphatidyl inositol (PI) linked to a cell membrane, but can be removed from membranes by treatment with a phosphatidyl inositol cleaving enzyme, e.g., phosphatidyl inositol phospholipase-C. This releases the antigen in a biologically active form, and allows purification by standard procedures of protein chemistry, See, e.g., Low (1989) Biochim. Biophys. Acta 988:427-454; Tse, et al. (1985) Science 230:1003-1008; and Brunner, et al. (1991) J. Cell Biol. 114:1275-1283.

Now that the DC-STAMP or DSP-1 has been characterized, fragments or derivatives thereof can be prepared by conventional processes for synthesizing peptides. These include processes such as are described in Stewart and Young (1984) Solid Phase Peptide Synthesis, Pierce Chemical Co., Rockford, Ill.; Bodanszky and Bodanszky (1984) The Practice of Peptide Synthesis, Springer-Verlag, New York; Bodanszky (1984) The Principles of Peptide Synthesis, Springer-Verlag, New York; and Villafranca (ed. 1991) Techniques in Protein Chemistry II, Academic Press, San Diego, Calif.

VIII. Uses

The present invention provides reagents which will find use in diagnostic applications as described elsewhere herein, e.g., in DC, T, NK, monocyte, or mast cell mediated conditions, or below in the description of kits for diagnosis. The gene may be useful in forensic sciences, e.g., to distinguish rodent from human, or as a marker to distinguish between different cells exhibiting differential expression or modification patterns.

This invention also provides reagents with significant commercial and/or therapeutic potential. The DC-STAMP or DSP-1 (naturally occurring or recombinant), fragments thereof, and antibodies thereto, along with compounds identified as having binding affinity to DC-STAMP or DSP-1, should be useful as reagents for teaching techniques of molecular biology, immunology, or physiology. Appropriate kits may be prepared with the reagents, e.g., in practical laboratory exercises in production or use of proteins, antibodies, cloning methods, histology, etc.

The reagents will also be useful in the treatment of conditions associated with abnormal physiology or development, including immunological conditions. They may be useful in vitro tests for presence or absence of interacting components, which may correlate with success of particular treatment strategies. In particular, modulation of physiology of various, e.g., hematopoietic or lymphoid, cells will be achieved by appropriate methods for treatment using the compositions provided herein. See, e.g., Thomson (ed. 1998) The Cytokine Handbook (3d ed.) Academic Press, San Diego; Metcalf and Nicola (1995) The Hematopoietic Colony Stimulating Factors Cambridge University Press; and Aggarwal and Gutterman (1991) Human Cytokines Blackwell Pub.

For example, a disease or disorder associated with abnormal expression or abnormal signaling by aPDC-STAMP should be a likely target for an agonist or antagonist. The new membrane proteins should play a role in regulation or development of hematopoietic cells, e.g., lymphoid cells, which affect immunological responses, e.g., inflammation and/or autoimmune disorders. Alternatively, it may affect vascular physiology or development, or neuronal effects.

In particular, the DC-STAMP is likely to-be important in mediating DC function. DC are the professional antigen presenting cells to T and B cells, and should be important in T cell mediated immune responses. Increases in T cell immunity should be important in, e.g., tumor immunotherapy, allergic conditions, and vaccine adjuvants. Important tumors include, e.g., carcinomas, including lung, colon, prostate, and breast, and melanomas. See, e.g., Bertino, et al. (eds. 1996) Encyclopedia of Cancer Academic Press; Devita, et al. (eds. 1997) Cancer: Principles & Practice of Oncology Lippincott, Williams and Wilkins; Devita (1997) Principles and Practice of Oncology Lippincott Williams and Wilkins; Cavalli, et al. (1996) Textbook of Medical Oncology Dunitz Martin Ltd; Horwich (ed. 1995) Oncology: A Multidisciplinary Textbook Lippincott-Raven; Peckham, et al. (eds. 1995) Oxford Textbook of Oncology Oxford Univ. Press; Mendelsohn, et al. (1995) The Molecular Basis of Cancer Saunders, Philadelphia; and McArdle (1990) Surgical Oncology: Current Concepts and Practice Butterworth-Heinemann. Among the allergic conditions, e.g., where a shift from Th2 humoral responses to Th1 cellular responses may be indicated, include asthma, pollen rhinitis, medicament allergies, food allergies, house dust mite allergies, etc. See, e.g., See, e.g., Lockey and Bukantz (eds. 1998) Allergen Immunotherapy; and Patterson (ed. 1997) Allergic Diseases: Diagnosis and Management. Conversely, decreases in T cell immunity should be important in, e.g., autoimmune conditions or transplantation rejection circumstances. Autoimmune diseases include, e.g., diabetes melitis, psoriasis, and multiple sclerosis. See, e.g., Morrow (ed. 1999) Autoimmune Rheumatic Disease, Weetman (ed. 1998) Endocrine Autoimmunity and Associated Conditions; Rose and Mackay (eds. 1998) The Autoimmune Diseases (3d ed.) Academic Press, San Diego; Kay (ed. 1997) Allergy and Allergic Diseases Blackwell Science, Malden Mass.; Samter, et al. (eds.) Immunological Diseases vols. 1 and 2, Little, Brown and Co.; and Coutinho and Kazatchkine (eds. 1993) Autoimmunity: Physiology and Disease. Transplant rejection and treatment are an established branch of medicine as described, e.g., in Racusen (ed. 1998) Kidney Transplant Rejection; Kelso and Clouston (1996) Cytokines in Transplantation; and Solez, et al. (eds. 1996) Solid Organ Transplant Rejection. Combination treatments might be used, combining a therapeutic related to the DC-STAMP or DSP-1 signaling with another therapeutic used to treat symptoms of the conditions, e.g., with Fit3 ligand, G, CSF, radiation or chemotherapy, antihistamines, IL-10, Treg1 cells, cyclosporin, or interferons.

Likewise, the DSP-1 therapeutic reagents may be useful to modulate function of monocyte, T, NK, or mast cell mediated conditions. It should be useful as a mast cell marker, being present on those cells, and likely modulates signaling with or by those cells.

Various abnormal conditions are known in different cell types which will produce DC-STAMP, e.g., as evaluated by mRNA expression by Northern blot analysis. See Berkow (ed.) The Merck Manual of Diagnosis and Therapy, Merck & Co., Rahway, N.J.; Thorn, et al. Harrison's Principles of Internal Medicine, McGraw-Hill, N.Y.; and Weatherall, et al. (eds.) Oxford Textbook of Medicine, Oxford University Press, Oxford. Many other medical conditions and diseases involve activation by T cells, and many of these will be responsive to treatment by an agonist or antagonist provided herein. See, e.g., Stites and Terr (eds.; 1991) Basic and Clinical Immunology Appleton and Lange, Norwalk, Connecticut; and Samter, et al. (eds.) Immunological Diseases Little, Brown and Co. These problems should be susceptible to prevention or treatment using compositions provided herein.

DC-STAMP or DSP-1, antagonists, antibodies, etc., can be purified and then administered to a patient, veterinary or human. These reagents can be combined for therapeutic use with additional active or inert ingredients, e.g., in conventional pharmaceutically acceptable carriers or diluents, e.g., immunogenic adjuvants, along with physiologically innocuous stabilizers, excipients, or preservatives. These combinations can be sterile filtered and placed into dosage forms as by lyophilization in dosage vials or storage in stabilized aqueous preparations. This invention also contemplates use of antibodies or binding fragments thereof, including forms which are not complement binding.

Drug screening using DC-STAMP or DSP-1 or fragments thereof can be performed to identify compounds having binding affinity to or other relevant biological effects on DC-STAMP or DSP-1 functions, including isolation of associated components. Subsequent biological assays can then be utilized to determine if the compound has intrinsic stimulating activity and is therefore a blocker or antagonist in that it blocks the activity of DC-STAMP or DSP-1 signaling. Likewise, a compound having intrinsic stimulating activity can activate the signal pathway and is thus an agonist in that it simulates the activity of DC-STAMP or DSP-1 signaling. Antibodies may be used to mediate, e.g., antigen dependent cell-mediated cytotoxicity, complement fixation, to localize enzymes or other means of activating inert pro-toxins, as diagnostic labels, or conjugated to compounds which will absorb energy to eliminate cells in proximity to where the antibody binds.

This invention further contemplates the therapeutic use of blocking antibodies to these antigens as antagonists and of stimulatory antibodies as agonists. This approach should be particularly useful with other DC-STAMP or DSP-1 species variants.

The quantities of reagents necessary for effective therapy-will depend upon many different factors, including means of administration, target site, physiological state of the patient, and other medicants administered. Thus, treatment dosages should be titrated to optimize safety and efficacy. Typically, dosages used in vitro may provide useful guidance in the amounts useful for in situ administration of these reagents. Animal testing of effective doses for treatment of particular disorders will provide further predictive indication of human dosage. Various considerations are described, e.g., in Gilman, et al. (eds.) Goodman and Gilman's: The Pharmacological Bases of Therapeutics, latest Ed., Pergamon Press; and Remington's Pharmaceutical Sciences, latest ed., Mack Publishing Co., Easton, Pa. Methods for administration are discussed therein and below, e.g., for oral, intravenous, intraperitoneal, or intramuscular administration, transdermal diffusion, and others. Pharmaceutically acceptable carriers will include water, saline, buffers, and other compounds described, e.g., in the Merck Index, Merck & Co., Rahway, N.J. Dosage ranges would ordinarily be expected to be in amounts lower than 1 mM concentrations, typically less than about 10 μM concentrations, usually less than about 100 nM, preferably less than about 10 pM (picomolar), and most preferably less than about 1 fM (femtomolar), with an appropriate carrier. Slow release formulations, or a slow release apparatus will often be utilized for continuous or long term administration. See, e.g., Langer (1990) Science 249:1527-1533.

DC-STAMP or DSP-1, fragments thereof, and antibodies to it or its fragments, antagonists, and agonists, may be administered directly to the host to be treated or, depending on the size of the compounds, it may be desirable to conjugate them to carrier proteins such as ovalbumin or serum albumin prior to their administration. Therapeutic formulations may be administered in many conventional dosage formulations. While it is possible for the active ingredient to be administered alone, it is preferable to present it as a pharmaceutical formulation. Formulations typically comprise at least one active ingredient, as defined above, together with one or more acceptable carriers thereof. Each carrier should be both pharmaceutically and physiologically acceptable in the sense of being compatible with the other ingredients and not injurious to the patient. Formulations include those suitable for oral, rectal, nasal, topical, or parenteral (including subcutaneous, intramuscular, intravenous and intradermal) administration. The formulations may conveniently be presented in unit dosage form and may be prepared by any methods well known in the art of pharmacy. See, e.g., Gilman, et al. (eds. 1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Pa.; Avis, et al. (eds. 1993) Pharmaceutical Dosage Forms: Parenteral Medications, Dekker, New York; Lieberman, et al. (eds. 1990) Pharmaceutical Dosage Forms: Tablets, Dekker, New York; and Lieberman, et al. (eds. 1990) Pharmaceutical Dosage Forms: Disperse Systems, Dekker, New York. The therapy of this invention may be combined with or used in association with other agents, e.g., other therapeutics for treatment of symptoms of the indications described.

Both naturally occurring and recombinant forms of the DC-STAMPs or DSP-1s of this invention are particularly useful in kits and assay methods which are capable of screening compounds for binding activity to the proteins. Several methods of automating assays have been developed in recent years so as to permit screening of tens of thousands of compounds in a short period. See, e.g., Fodor, et al. (1991) Science 251:767-773, which describes means for testing of binding affinity by a plurality of defined polymers synthesized on a solid substrate. The development of suitable assays can be greatly facilitated by the availability of large amounts of purified, soluble antigens as provided by this invention.

Other methods can be used to determine the critical residues in DC-STAMP or DSP-1 counterreceptor or ligand interactions. Mutational analysis can be performed, e.g., see Somoza, et al. (1993) J. Exptl. Med. 178:549-558, to determine specific residues critical in the interaction and/or signaling. PHD (Rost and Sander (1994) Proteins 19:55-72) and DSC (King and Sternberg (1996) Protein Sci. 5:2298-2310) can provide secondary structure predictions of α-helix (H), β-strand (E), or coil (L). Surface exposed residues would affect ligand or receptor binding, while embedded residues would affect general structure.

For example, antagonists can normally be found once the antigen has been structurally defined, e.g., by tertiary structure data. Testing of potential interacting analogs is now possible upon the development of highly automated assay methods using a purified DC-STAMP or DSP-1. In particular, new agonists and antagonists will be discovered by using screening techniques described herein. Of particular importance are compounds found to have a combined binding affinity for a spectrum of DC-STAMP molecules, e.g., compounds which can serve as antagonists for species variants of DC-STAMP.

One method of drug screening utilizes eukaryotic or prokaryotic host cells which are stably transformed with recombinant DNA molecules expressing a DC-STAMP or DSP-1. Cells may be isolated which express an antigen in isolation from other molecules. Such cells, either in viable or fixed form, can be used for standard binding partner binding assays. See also, Parce, et al. (1989) Science 246:243-247; and Owicki, et al. (1990) Proc. Nat'l Acad. Sci. USA 87:4007-4011, which describe sensitive methods to detect cellular responses.

Another technique for drug screening involves an approach which provides high throughput screening for compounds having suitable binding affinity to an antigen and is described in detail in Geysen, European Patent Application 84/03564, published on Sep. 13, 1984. First, large numbers of different small peptide test compounds are synthesized on a solid substrate, e.g., plastic pins or some other appropriate surface, see Fodor, et al. (1991). Then all the pins are reacted with solubilized, unpurified or solubilized, purified DC-STAMP, and washed. The next step involves detecting bound DC-STAMP.

Rational drug design may also be based upon structural studies of the molecular shapes of the DC-STAMP or DSP-1 and other effectors or analogs. Effectors may be other proteins which mediate other functions in response to binding, or other proteins which normally interact with the membrane proteins, e.g., a receptor. One means for determining which sites interact with specific other proteins is a physical structure determination, e.g., x-ray crystallography or 2 dimensional NMR techniques. These will provide guidance as to which amino acid residues form molecular contact regions, as modeled, e.g., against other cytokine-receptor models. For a detailed description of protein structural determination, see, e.g., Blundell and Johnson (1976) Protein Crystallography, Academic Press, New York.

IX. Kits

This invention also contemplates use of DC-STAMP or DSP-1 proteins, fragments thereof, peptides, and their fusion products in a variety of diagnostic kits and methods for detecting the presence of another DC-STAMP or DSP-1 or binding partner. Typically the kit will have a compartment containing either a defined DC-STAMP or DSP-1 peptide or gene segment or a reagent which recognizes one or the other, e.g., DC-STAMP or DSP-1 fragments or antibodies.

A kit for determining the binding affinity of a test compound to a DC-STAMP would typically comprise a test compound; a labeled compound, for example a binding partner or antibody having known binding affinity for DC-STAMP; a source of DC-STAMP (naturally occurring or recombinant); and a means for separating bound from free labeled compound, such as a solid phase for immobilizing the molecule. Compartments containing reagents, and instructions, will normally be provided. Once test compounds are screened, those having suitable binding affinity to the antigen can be evaluated in suitable biological assays, as are well known in the art, to determine whether they act as agonists or antagonists to DC-STAMP signaling pathway. The availability of recombinant DC-STAMP polypeptides also provides well defined standards for calibrating such assays.

Antibodies, including antigen binding fragments, specific for the DC-STAMP or DSP-1 or fragments are useful in diagnostic applications to detect the presence of elevated levels of antigen and/or its fragments. Such diagnostic assays can employ lysates, live cells, fixed cells, immunofluorescence, cell cultures, body fluids, and further can involve the detection of antigens related to the antigen in serum, or the like. Diagnostic assays may be homogeneous (without a separation step between free reagent and antigen-binding partner complex) or heterogeneous (with a separation step). Various commercial assays exist, such as radioimmunoassay (RIA), enzyme-linked immunosorbent assay (ELISA), enzyme immunoassay (EIA), enzyme-multiplied immunoassay technique (EMIT), substrate-labeled fluorescent immunoassay (SLFIA), and the like. See, e.g., Van Vunakis, et al. (1980) Meth Enzymol. 70:1-525; Harlow and Lane (1980) Antibodies: A Laboratory Manual, CSH Press, NY; and Coligan, et al. (eds. 1993) Current Protocols in Immunology, Greene and Wiley, NY.

Anti-idiotypic antibodies may have similar use to diagnose presence of antibodies against a DC-STAMP or DSP-1, as such may be diagnostic of various abnormal states. For example, overproduction of DSP-1 may result in production of various immunological reactions which may be diagnostic of abnormal physiological states, particularly in allergic conditions.

Frequently, the reagents for diagnostic assays are supplied in kits, so as to optimize the sensitivity of the assay. For the subject invention, depending upon the nature of the assay, the protocol, and the label, either labeled or unlabeled antibody or binding partner, or labeled antigen is provided. This is usually in conjunction with other additives, such as buffers, stabilizers, materials necessary for signal production such as substrates for enzymes, and the like. Preferably, the kit will also contain instructions for proper use and disposal of the contents after use. Typically the kit has compartments for each useful reagent. Desirably, the reagents are provided as a dry lyophilized powder, where the reagents may be reconstituted in an aqueous medium providing appropriate concentrations of reagents for performing the assay.

Many of the aforementioned constituents of the drug screening and the diagnostic assays may be used without modification or may be modified in a variety of ways. For example, labeling may be achieved by covalently or non-covalently joining a moiety which directly or indirectly provides a detectable signal. In any of these assays, the binding partner, test compound, antigen, or antibodies thereto can be labeled either directly or indirectly. Possibilities for direct labeling include label groups: radiolabels such as ¹²⁵I, enzymes (U.S. Pat. No. 3,645,090) such as peroxidase and alkaline phosphatase, and fluorescent labels (U.S. Pat. No. 3,940,475) capable of monitoring the change in fluorescence intensity, wavelength shift, or fluorescence polarization. Possibilities for indirect labeling include biotinylation of one constituent followed by binding to avidin coupled to one of the above label groups.

There are also numerous methods of separating the bound from the free antigen, or alternatively the bound from the free test compound. The antigen can be immobilized on various matrixes followed by washing. Suitable matrixes include plastic such as an ELISA plate, filters, and beads. See, e.g., Coligan, et al. (eds. 1993) Current Protocols in Immunology, Vol. 1, Chapter 2, Greene and Wiley, NY. Other suitable separation techniques include, without limitation, the fluorescein antibody magnetizable particle method described in Rattle, et al. (1984) Clin. Chem. 30:1457-1461, and the double antibody magnetic particle separation as described in U.S. Pat. No. 4,659,678.

Methods for linking proteins or their fragments to the various labels are well known. Many of the techniques involve the use of activated carboxyl groups either through the use of carbodiimide or active esters to form peptide bonds, the formation of thioethers by reaction of a mercapto group with an activated halogen such as chloroacetyl, or an activated olefin such as maleimide, for linkage, or the like. Fusion proteins will also find use in these applications.

Another diagnostic aspect of this invention involves use of oligonucleotide or polynucleotide sequences taken from the sequence of a DC-STAMP or DSP-1. These sequences can be used as probes for detecting levels of the antigen message in samples from patients suspected of having an abnormal condition, e.g., inflammatory or autoimmune. The preparation of both RNA and DNA nucleotide sequences, the labeling of the sequences, and the preferred size of the sequences has received ample description and discussion in the literature. See, e.g., Langer-Safer, et al. (1982) Proc. Nat'l. Acad. Sci. 79:4381-4385; Caskey (1987) Science 236:962-967; and Wilchek et al. (1988) Anal. Biochem. 171:1-32.

Diagnostic kits which also test for the qualitative or quantitative expression of other molecules are also contemplated. Diagnosis or prognosis may depend on the combination of multiple indications used as markers. Thus, kits may test for combinations of markers. See, e.g., Viallet, et al. (1989) Progress in Growth Factor Res. 1:89-97. Kits may contain additional reagents to evaluate other cell subsets.

X. Isolating a DC-STAMP or DSP-1 Ligand or Receptor

Both DC-STAMP and DSP-1 are cell surface antigens, which may be receptor for a ligand or another surface antigen. Having isolated one component of such an interaction, methods exist for isolating a ligand or binding receptor partner. See, Gearing, et al. (1989) EMBO J. 8:3667-3676. For example, means to label the antigen without interfering with the binding to its partner can be determined. For example, an affinity label can be fused to either the amino- or carboxyl-terminus of the ligand. Such label may be a FLAG epitope tag, or, e.g., an Ig or Fc domain. An expression library can be screened for specific binding of the antigen, e.g., by cell sorting, or other screening to detect subpopulations which express such a binding component. See, e.g., Ho, et al. (1993) Proc. Nat'l Acad. Sci. USA 90:11267-11271; and Liu, et al. (1994) J. Immunol. 152:1821-29. Alternatively, a panning method may be used. See, e.g., Seed and Aruffo (1987) Proc. Nat'l Acad. Sci. USA 84:3365-3369.

Protein cross-linking techniques with label can be applied to isolate binding partners of the DC-STAMP or DSP-1. This would allow identification of proteins which specifically interact with the antigen, e.g., in a ligand-receptor or receptor-receptor manner.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

### EXAMPLES

I. General Methods

Many of the standard methods below are described or referenced, e.g., in Maniatis, et al. (1982) Molecular Cloning, A Laboratory Manual Cold Spring Harbor Laboratory, Cold Spring Harbor Press, NY; Sambrook, et al. (1989) Molecular Cloning: A Laboratory Manual (2d ed.) Vols. 1-3, CSH Press, NY; Ausubel, et al., Biology Greene Publishing Associates, Brooklyn, N.Y.; or Ausubel, et al. (1987 and Supplements) Current Protocols in Molecular Biology Wiley/Greene, NY; Innis, et al. (eds. 1990) PCR Protocols: A Guide to Methods and Applications Academic Press, NY. Methods for protein purification include such methods as ammonium sulfate precipitation, column chromatography, electrophoresis, centrifugation, crystallization, and others. See, e.g., Ausubel, et al. (1987 and periodic supplements); Deutscher (1990) “Guide to Protein Purification,” Methods in Enzymology vol. 182, and other volumes in this series; Coligan, et al. (1995 and supplements) Current Protocols in Protein Science John Wiley and Sons, New York, N.Y.; P. Matsudaira (ed. 1993) A Practical Guide to Protein and Peptide Purification for Microsequencing, Academic Press, San Diego, Calif.; and manufacturer's literature on use of protein purification products, e.g., Pharmacia, Piscataway, N.J., or Bio-Rad, Richmond, Calif. Combination with recombinant techniques allow fusion to appropriate segments (epitope tags), e.g., to a FLAG sequence or an equivalent which can be fused, e.g., via a protease-removable sequence. See, e.g., Hochuli (1989) Chemische Industrie 12:69-70; Hochuli (1990) “Purification of Recombinant Proteins with Metal Chelate Absorbent” in Setlow (ed.) Genetic Engineering, Principle and Methods 12:87-98, Plenum Press, NY; and Crowe, et al. (1992) QIAexpress: The High Level Expression & Protein Purification System QUIAGEN, Inc., Chatsworth, Calif.

Standard immunological techniques are described, e.g., in Hertzenberg, et al. (eds. 1996) Weir's Handbook of Experimental Immunology vols. 1-4, Blackwell Science; Coligan (1991) Current Protocols in Immunology Wiley/Greene, NY; and Methods in Enzymology vols. 70, 73, 74, 84, 92, 93, 108, 116, 121, 132, 150, 162, and 163. Cytokine assays are described, e.g., in Thomson (ed. 1998) The Cytokine Handbook (3d ed.) Academic Press, San Diego; Mire-Sluis and Thorpe (1998) Cytokines Academic Press, San Diego; Metcalf and Nicola (1995) The Hematopoietic Colony Stimulating Factors Cambridge University Press; and Aggarwal and Gutterman (1991) Human Cytokines Blackwell Pub.

Assays for vascular biological activities are well known in the art. They will cover angiogenic and angiostatic activities in tumor, or other tissues, e.g., arterial smooth muscle proliferation (see, e.g., Koyoma, et al. (1996) Cell 87:1069-1078), monocyte adhesion to vascular epithelium (see McEvoy, et al. (1997) J. Exp. Med. 185:2069-2077), etc. See also Ross (1993) Nature 362:801-809; Rekhter and Gordon (1995) Am. J. Pathol. 147:668-677; Thyberg, et al. (1990) Atherosclerosis 10:966-990; and Gumbiner (1996) Cell 84:345-357.

Assays for neural cell biological activities are described, e.g., in Wouterlood (ed. 1995) Neuroscience Protocols modules 10, Elsevier; Methods in Neurosciences Academic Press; and Neuromethods Humana Press, Totowa, N.J. Methodology of developmental systems is described, e.g., in Meisami (ed.) Handbook of Human Growth and Developmental Biology CRC Press; and Chrispeels (ed.) Molecular Techniques and Approaches in Developmental Biology Interscience.

FACS analyses are described in Melamed, et al. (1990) Flow Cytometry and Sorting Wiley-Liss, Inc., New York, N.Y.; Shapiro (1988) Practical Flow Cytometry Liss, New York, N.Y.; and Robinson, et al. (1993) Handbook of Flow Cytometry Methods Wiley-Liss, New York, N.Y.

II. Cloning of Human DC-STAMP and DSP-1

The sequence of the primate, e.g., human, DC-STAMP gene is provided in Table 1. The sequence is derived from sequence of a cDNA clone isolated from dendritic cells. This sequence allows preparation of PCR primers, or probes, to determine cellular distribution of the gene. The sequence allows isolation of genomic DNA which encode the message.

The ORF of DC-STAMP predicts a protein of 470 amino acids, with a predicted molecular weight of around 53 kD and an isoelectric point of 9.41. The amino terminus of the protein starts with a short stretch of hydrophobic amino acids, which predicts an uncleavable signal sequence (PSORT, Osaka University, Japan). Hydrophobicity analysis of the sequence revealed 5 strong and 2 weak hydrophobic stretches of 18-20 amino acids, suggesting that the DC-STAMP molecule is spanning the membrane multiple times. The TM Predict program from BCM Search Launcher (K. Hofman and W. Hofman) suggests a topology model in which the DC-STAMP protein contains 7 transmembrane spanning regions, with the N-terminus located outside and the C-terminus on the luminal side of the membrane.

Interestingly, the DC-STAMP protein contains two pairs of cysteine residues, one at the start of the first transmembrane domain (TM1), the other at the end of the second transmembrane domain (TM2). These cysteines might form a disulphide bridge near the outer side of the membrane, and stabilize the protein structure. Prosite analysis of the protein revealed 3 potential glycosylation sites, two on the second and one on the third putative extracellular loop. In addition, there is a consensus sequence for phosphorylation by protein kinase C between the fifth and sixth transmembrane region, which is the second intracellular loop according to the proposed topology. The 72 amino acid cytoplasmic tail of DC-STAMP contains several serine residues, two of which might serve as a target for phosphorylation. Interestingly, the C-terminus of the DC-STAMP protein is surprisingly rich in positively charged residues, comprising 25% of the tail and conferring an overall positive charge (+14).

The DSP-1 gene was isolated from a cDNA library made from human HEL cells.

Using the probe or PCR primers, various tissues or cell types are probed to determine cellular distribution. PCR products are cloned using, e.g., a TA cloning kit (Invitrogen). The resulting cDNA plasmids are sequenced from both termini on an automated sequencer (Applied Biosystems).

Leukocyte Preparations

PBMC were obtained by leukophoresis of blood from healthy donors, and adherence for 2 hours resulted in a non-adherent PBL fraction. Monocytes were elutriated from PBMC by counterflow centrifugation, resulting in a population of cells that were greater than 85% CD14⁺. Fractions were cultured in Iscove's medium supplemented with 5% FCS and 1% antibiotics/antimyotics (Life Technologies Inc., Grand Island, N.Y.). Both the non-adherent PBL and total PBMC were stimulated with phytohemagglutinin (PHA; 1 μg/ml; Murex Diagnostics Ltd, Dartford, England) and rIL-2 (200 U/ml; Cetus Corp., Emeryville, Calif.) for 16 h. Elutriated monocytes were stimulated with 2 μg/ml LPS for 16 h.

DC were generated in vitro from monocytes using a modification of described methods. See Ridge, et al. (1998) Nature 393:474-478; and Bennett, et al. (1998) Nature 393:478-480. Monocytes were cultured in AIM-V medium (Life Technologies Ltd, Paisley, Scotland) supplemented with 5% fetal calf serum and in the presence of 800 U/ml GM-CSF and 500 U/ml IL-4 (both from Schering-Plough, The Netherlands) for 5-7 days. Resulting DC were collected directly or after activation with either LPS for 16 h (2 μg/ml), or after the sequential addition of TNFα (10 ng/ml, 24 h) and the activating anti-CD40 antibody MAB89, generously provided by DNAX, Palo Alto, Calif. (1.5 μg/ml, 24 h). Purified tonsil B lymphocytes were isolated according to the method described by Falkoff, et al. (1982). J. Immunol. Methods 50:39-49.

cDNA Library Preparation

Complementary DNA libraries were prepared. See Marland, et al. (1997) in Ricciardi-Castognoli (ed.) Dendritic Cells in Fundamental and Clinical Immunology, Vol 3, Plenum Publ. Corporation; and Adema, et al. (1997) Nature 387:713-717. Nucleotide sequences were analyzed against the non-redundant GenBank and EMBL databases using the BLAST program. See Altschul, et al. (1990) J. Mol. Biol. 215:403-410.

Northern Blot Analysis for DC-STAMP

Total RNA was isolated from DC cultures; e.g., monocyte-derived DC cultured for 7 days in IL-4 and GM-CSF, freshly isolated leukocytes and cell lines using the guanidine thiocyanate/cesium chloride procedure. Poly(A)⁺ RNA was isolated from the DC fraction by affinity chromatography (Oligotex, Qiagen). Per sample 20 μg total RNA or 2 μg poly(A) RNA was resolved overnight on a formaldehyde gel and transferred to a nylon membrane by capillary blotting. Hybridization was performed overnight at 65° C. in Church solution (0.5 M NaHPO₄, pH 7.2; 7% SDS; 0.5 M EDTA). Multiple tissue Northern blot #7780-1 (Clontech, Palo Alto, Calif.) was probed and washed under stringent conditions according to the manufacturer's recommendations. Both Northern blots were probed with the 444 bp SalI-RcaI fragment, comprising part of the 3′ UTR of DC-STAMP, randomly labeled with ³²P (T7 QuickPrime Kit, Pharmacia).

RT-PCR

Total RNA was isolated using Trizol Reagent (Gibco BRL) and treated with RNAse free DNAse (Boehringer Mannheim). 1 μg RNA was transcribed into cDNA using an oligodT primer and Superscript II reverse transcriptase (RT, Gibco BRL). Half of the cDNA was used to amplify the DC-STAMP message, according to a standard PCR protocol (24 cycles). The primers were located in the most 3′ part of the DC-STAMP. ORF, yielding a specific product of 334 bp. As a control for RNA quality, the other half of the cDNA was used to amplify a β actin product of 328 bp (18 cycles). Southern blot analysis of the PCR products was performed using an ³²P-end labeled internal oligonucleotide (Klenow polymerase, Boehringer Mannheim) from either DC-STAMP or actin. Samples without RT were always completely negative.

Library Screening and 5′ RACE PCR

100,000 colonies from a cDNA library derived from non-stimulated DC were screened using the randomly labeled 444 bp RcaI/SalI fragment from the original 936 bp cDNA clone as a probe. The most 5′ end of the DC-STAMP cDNA was isolated by 5′ RACE PCR. Briefly, 1 μg of total DC RNA was transcribed into cDNA (Superscript II Reverse Transcriptase, Gibco BRL), using a DC-STAMP specific 5′ RACE-1 primer. The cDNA was purified with a QIAQuick PCR purification kit (Qiagen) and subsequently tailed using 50 U of Terminal Transferase (Boehringer Mannheim) in the presence of dCTP (5 gM) and 0.75 mM COCl₂ (15 minutes 37° C.). The tailed cDNA was extracted once with phenol/chloroform and precipitated using glycogen (50 μg). 5% of the tailed cDNA was used in a hemi-nested PCR reaction, using nested DC-STAMP specific primer 5′RACE-2 and a 5′ primer annealing to the C-tail of the cDNA. 30 PCR cycles were performed using a standard program (1 min 94° C., 1 min 58° C., 1 min 72° C., 10 min extension at 72° C.). The resulting PCR product was gel-purified and cloned into the TA-cloning vector PGEM-T (Promega). The overlapping cDNA fragments were sequenced by the dideoxy chain reaction (AutoRead Sequencing kit, Pharmacia Biotech) on the ALF Express automated sequencer (Pharmacia Biotech). The complete ORF of DC-STAMP was amplified from oligo dT transcribed cDNA (Superscript II, Gibco BRL) using the Expand Long Template PCR System (30 cycles, Boehringer Mannheim) and cloned into pGEM-T Easy. Sequence analysis of several clones confirmed the sequence obtained by 5′RACE PCR.

DC-STAMP cDNA Encodes a 470 Amino Acid Multimembrane Spanning Molecule

Since expression of DC-STAMP was specifically detected in DC, a DC cDNA library was screened with a specific probe for DC-STAMP in order to obtain the full-length transcript. Several DC-STAMP cDNA clones were isolated, of which the longest clone contained an insert of 1.4 kb, identical to the original clone at its 3′ end. 5′ RACE PCR resulted in the cloning of the most 5′ region of the DC-STAMP messenger. Northern blot analysis using this 5′ DC-STAMP fragment as a probe resulted in the same 2.3 kb message as described, indicating that both fragments belong to a single cDNA. The cDNA encoding DC-STAMP has a total length of 1954 bp, which nicely fits with the 2.3 kb messenger on Northern blot, suggesting a poly A tail of around 350 bp. It contains a single long ORF of 1410 nucleotides starting with the first ATG codon at nucleotide 52, which is in the appropriate sequence context for translation initiation (Kozak (1987) Nucleic Acid. Res. 15:8125-8148), and is followed by a 490-nucleotide 3′ UTR. The poly A-tail is preceded by the polyadenylation signal sequence ATTAAA. See Table 1.

Comparison of the DC-STAMP amino acid and nucleotide sequence with known sequences in the GenBank/EMBL databases revealed no homology, except two nucleotide matches with unpublished EST fragments in the dbEST, derived from human skin tumor and human neuroendocrine lung carcinoid (accession numbers AA380009 and AI268407, respectively).

The sequence of the primate, e.g., human, DSP-1 gene is provided in Table 2. The sequence is derived from sequence of a cDNA clone isolated human HEL cells.

III. Cellular Expression of Antigen

An appropriate probe or primers specific for cDNA encoding primate antigen are prepared. Typically, the probe is labeled, e.g., by random priming.

Southern Analysis: DNA (5 μg) from a primary amplified cDNA library was digested with appropriate restriction enzymes to release the inserts, run on a 1% agarose gel and transferred to a nylon membrane (Schleicher and Schuell, Keene, N.H.).

Samples for human mRNA isolation may include: peripheral blood mononuclear cells (monocytes, T cells, NK cells, granulocytes, B cells), resting (T100); peripheral blood mononuclear cells, activated with anti-CD3 for 2, 6, 12 h pooled (T101); T cell, THO clone Mot 72, resting (T102); T cell, THO clone Mot 72, activated with anti-CD28 and anti-CD3 for 3, 6, 12 h pooled (T103); T cell, THO clone Mot 72, anergic treated with specific peptide for 2, 7, 12 h pooled (T104); T cell, TH1 clone HY06, resting (T107); T cell, TH1 clone HY06, activated with anti-CD28 and anti-CD3 for 3, 6, 12 h pooled (T108); T cell, TH1 clone HY06, anergic treated with specific peptide for 2, 6, 12 h pooled (T109); T cell, TH2 clone HY935, resting (T110); T cell, TH2 clone HY935, activated with anti-CD28 and anti-CD3 for 2, 7, 12 h pooled (T111); T cell tumor lines Jurkat and Hut78, resting (T117); T cell clones, pooled AD130.2, Tc783.12, Tc783.13, Tc783.58, Tc782.69, resting (T118); T cell random γδ T cell clones, resting (T119); CD28− T cell clone; Splenocytes, resting (B100); Splenocytes, activated with anti-CD40 and IL-4 (B101); B cell EBV lines pooled WT49, RSB, JY, CVIR, 721.221, RM3, HSY, resting (B102); B cell line JY, activated with PMA and ionomycin for 1, 6 h pooled (B103); NK 20 clones pooled, resting (K100); NK 20 clones pooled, activated with PMA and ionomycin for 6 h (K101); NKL clone, derived from peripheral blood of LGL leukemia patient, IL-2 treated (K106); hematopoietic precursor line TF1, activated with PMA and ionomycin for 1, 6 h pooled (C100); U937 premonocytic line, resting (M100); U937 premonocytic line, activated with PMA and ionomycin for 1, 6 h pooled (M101); elutriated monocytes, activated with LPS, IFNγ, anti-IL-10 for 1, 2, 6, 12, 24 h pooled (M102); elutriated monocytes, activated with LPS, IFNγ, IL-10 for 1, 2, 6, 12, 24 h pooled (M103); elutriated monocytes, activated with LPS, IFNγ, anti-IL-10 for 4, 16 h pooled (M106); elutriated monocytes, activated with LPS, IFNγ, IL-10 for 4, 16 h pooled (M107); elutriated monocytes, activated LPS for 1 h (M108); elutriated monocytes, activated LPS for 6 h (M109); DC 70% CD1a+, from CD34+ GM-CSF, TNFα 12 days, resting (D101); DC 70% CD1a+, from CD34+ GM-CSF, TNFα 12 days, activated with PMA and ionomycin for 1 hr (D102); DC 70% CD1a+, from CD34+ GM-CSF, TNFα 12 days, activated with PMA and ionomycin for 6 hr (D103); DC 95% CD1a+, from CD34+ GM-CSF, TNFα 12 days FACS sorted, activated with PMA and ionomycin for 1, 6 h pooled (D104); DC 95% CD14+, ex CD34+ GM-CSF, TNFα 12 days FACS sorted, activated with PMA and ionomycin 1, 6 hr pooled (D105); DC CD1a+CD86+, from CD34+ GM-CSF, TNFα 12 days FACS sorted, activated with PMA and ionomycin for 1, 6 h pooled (D106); DC from monocytes GM-CSF, IL-4 5 days, resting (D107); DC from monocytes GM-CSF, IL-4 5 days, resting (D108); DC from monocytes GM-CSF, IL-4 5 days, activated LPS 4, 16 h pooled (D109); DC from monocytes GM-CSF, IL-4 5 days, activated TNFα, monocyte supe for 4, 16 h pooled (D110); epithelial cells, unstimulated; epithelial cells, IL-11 activated; lung fibroblast sarcoma line MRC5, activated with PMA and ionomycin for 1, 6 h pooled (C101); kidney epithelial carcinoma cell line CHA, activated with PMA and ionomycin for 1, 6 h pooled (C102).

Rodent counterparts, e.g., mouse, should be identified, and their distributions will be similarly evaluated. Samples for mouse mRNA isolation can include: resting mouse fibroblastic L cell line (C200); Braf:ER (Braf fusion to estrogen receptor) transfected cells, control (C201); Mel14+ naive T cells from spleen, resting (T209); Mel14+ naive T cells from spleen, stimulated with IFNγ, IL-12, and anti IL-4 to polarize to TH1 cells, exposed to IFNγ and IL-4 for 6, 12, 24 h, pooled (T210); Mel14+ naive T cells from spleen, stimulated with IL-4 and anti IFNγ to polarize to Th2 cells, exposed to IL-4 and anti IFNγ for 6, 13, 24 h, pooled (T211); T cells, TH1 polarized (Mel14 bright, CD4+ cells from spleen, polarized for 7 days with IFN-γ and anti IL-4; T200); T cells, TH2 polarized (Mel14 bright, CD4+ cells from spleen, polarized for 7 days with IL-4 and anti-IFN-γ; T201); T cells, highly TH1 polarized 3× from transgenic Balb/C (see Openshaw, et al. (1995) J. Exp. Med. 182:1357-1367; activated with anti-CD3 for 2, 6, 24 h pooled; T202); T cells, highly TH2 polarized 3× from transgenic Balb/C (activated with anti-CD3 for 2, 6, 24 h pooled (T203); T cells, highly TH1 polarized 3× from transgenic C57 bl/6 (activated with anti-CD3 for 2, 6, 24 h pooled; T212); T cells, highly TH2 polarized 3× from transgenic C57 bl/6 (activated with anti-CD3 for 2, 6, 24 h pooled; T213); T cells, highly TH1 polarized (naive CD4+ T cells from transgenic Balb/C, polarized 3× with IFNγ, IL-12, and anti-IL-4; stimulated with IGIF, IL-12, and anti IL-4 for 6, 12, 24 h, pooled); CD44− CD25+ pre T cells, sorted from thymus (T204); TH1 T cell clone D1.1, resting for 3 weeks after last stimulation with antigen (T205); TH1 T cell clone D1.1, 10 μg/ml ConA stimulated 15 h (T206); TH2 T cell clone CDC35, resting for 3 weeks after last stimulation with antigen (T207); TH2 T cell clone CDC35, 10 μg/ml-ConA stimulated 15 h (T208); unstimulated B cell line CH12 (B201); unstimulated mature B cell leukemia cell line A20 (B200); unstimulated large B cells from spleen (B202); B cells from total spleen, LPS activated (B203); metrizamide enriched dendritic cells from spleen, resting (D200); dendritic cells from bone marrow, resting (D201); unstimulated bone marrow derived dendritic cells depleted with anti B220, anti CD3, and anti Class II, cultured in GM-CSF and IL-4 (D202); bone marrow derived dendritic cells depleted with anti B220, anti CD3, and anti Class II, cultured in GM-CSF and IL-4, stimulated with anti CD40 for 1, 5 d, pooled (D203); monocyte cell line RAW 264.7 activated with LPS 4 h (M200); bone-marrow macrophages derived with GM and M-CSF (M201); bone-marrow macrophages derived with GM-CSF, stimulated with LPS, IFNγ, and IL-10 for 24 h (M205); bone-marrow macrophages derived with GM-CSF, stimulated with LPS, IFNγ, and anti IL-10 for 24 h (M206); peritoneal macrophages (M207); macrophage cell line J774, resting (M202); macrophage cell line J774+LPS+anti-IL-10 at 0.5, 1, 3, 6, 12 h pooled (M203); macrophage cell line J774+LPS+IL-10 at 0.5, 1, 3, 5, 12 h pooled (M204); unstimulated mast cell lines MC-9 and MCP-12 (M208); immortalized endothelial cell line derived from brain microvascular endothelial cells, unstimulated (E200); immortalized endothelial cell line derived from brain microvascular endothelial cells, stimulated overnight with TNFα (E201); immortalized endothelial cell line derived from brain microvascular endothelial cells, stimulated overnight with TNFα (E202); immortalized endothelial cell line derived from brain microvascular endothelial cells, stimulated overnight with TNFα and IL-10 (E203); total aorta from wt C57 bl/6 mouse; total aorta from 5 month ApoE KO mouse (X207); total aorta from 12 month ApoE KO mouse (X207); wt thymus (0214); total thymus, rag-1 (0208); total kidney, rag-1 (0209); total kidney, NZ B/W mouse; and total heart, rag-1 (0202).

To further analyze the expression pattern of DC-STAMP, RT-PCR was performed on RNA from a panel of freshly isolated resting or activated leukocyte populations and several cell lines of haematopoietic as well as non-haematopoietic origin. The PCR products were Southern blotted and hybridized with a specific DC-STAMP oligonucleotide. A distinct band of the expected size was detected in immature as well as in mature DC, stimulated with either LPS or a combination of TNFα and an activating anti-CD40 antibody. In contrast, freshly isolated monocytes did not express the DC-STAMP RNA, even after overnight stimulation with LPS. A low expression was detected in total PBMC, which could be explained by the presence of contaminating DC, and in the pre-monocytic cell line U937. The β-actin mRNA control was similar in all samples, indicating that equal amounts of RNA were used for the RT-PCR.

Hybridization of a Northern blot containing MRNA from 12 human tissues (Clontech MTN #7780-1) with a DC-STAMP specific probe did not result in any detectable signal, not even after exposure of the blot for several days. The absence of detectable expression of DC-STAMP in the 12 different human tissues tested is consistent with a relatively low expression in DC.

Initial distribution studies of the DSP-1 suggest that the antigen is primarily expressed on monocytes, mast cells, T cells, and NK cells. Thus, it is likely that the receptor has a negative regulatory role for those cell types.

IV. Fusion Protein Constructs

The DC-STAMP ORF was amplified with Pwo DNA polymerase (Boehringer Mannheim) using appropriate primers, containing a EcoRI or BglII site, respectively, deleting the DC-STAMP STOP codon. This PCR product was cloned as an EcoRI/BglII fragment into the pN3-EGFP expression vector (Clontech, Palo Alto, Calif.), digested with EcoRI and BamHI, inserting the DC-STAMP cDNA N-terminal of the transcript encoding the enhanced green fluorescent protein (EGFP). The CCR1 molecule was amplified by RT-PCR using total RNA from monocytes and primers based on the published sequence (see Adema, et al. (1997) Nature 387:713-717; and Falkoff, et al. (1982). J. Immunol. Methods 50:39-49) and cloned as a GFP fusion protein using a similar approach. Primers contained a NotI or BamHI restriction site. The digested PCR product was cloned into the NotI-BamHI digested pBluescript SKI-vector (Stratagene, La Jolla, Calif.) and subsequently cloned as a SacI-BamHI fragment into the expression vector pN3-EGFP.

Similar constructs can be made using the DSP-1 sequences.

V. Cellular Localization of DC-STAMP

To determine the cellular localization of DC-STAMP, the DC-STAMP-EGFP (Enhanced Green Fluorescent Protein) fusion protein was subjected to analysis. This construct possessed the EGFP sequence fused to the C-terminal of the DC-STAMP ORF. 293 cells were transfected with this construct, and analyzed by Confocal Laser Scan Microscopy (CLSM). Since multimembrane spanning proteins are very hydrophobic and complex proteins, the CCR1 molecule, a 7 TM chemokine receptor expressed at the cell membrane, was compared as a control. Transfection of CCR1-EGFP into 293 cells resulted in a bright membrane fluorescence, often accompanied by an additional highly fluorescent spot in the cytoplasm, possibly representing the Golgi. Analysis of transient as well as stable transfectants of the DC-STAMP-EGFP construct showed a similar fluorescence staining pattern as seen for CCR1-EGFP, indicating that DC-STAMP can also be expressed at the cell surface. Transfectants expressing the EGFP protein alone showed a bright cytoplasmic fluorescence, not localized to a particular cell structure.

The localization of the C-terminus of the DC-STAMP-EGFP protein was determined by staining the DC-STAMP-EGFP transient transfectants with polyclonal anti-GFP serum either before or after permeabilization. Cytospin stainings showed that EGFP could only be detected after permeabilization, indicating that DC-STAMP has an intracellular C-terminus. The amount of positive cells was consistent with the percentage of GFP positive cells in the transient transfected bulk population as observed by FACS analysis (30%). The few cells that stained positive after pre-incubation with the anti-GFP serum were due to leakage of the antibody into dead cells.

VI. Chromosome Mapping of DC-STAMP and DSP-1

An isolated cDNA encoding the antigen is used. Chromosome mapping is a standard technique. See, e.g., BIOS Laboratories (New Haven, Conn.) and methods for using a mouse somatic cell hybrid panel with PCR.

VII. Purification of DC-STAMP or DSP-1 Protein

Multiple transfected cell lines are screened for one which expresses the desired antigen at a high level compared with other cells. Various cell lines are screened and selected for their favorable properties in handling. Natural antigen can be isolated from natural sources, or by expression from a transformed cell using an appropriate expression vector. Purification of the expressed protein is achieved by standard procedures, or may be combined with engineered means for effective purification at high efficiency from cell lysates or supernatants. FLAG or His6 segments can be used for such purification features. Alternatively, affinity chromatography may be used with specific antibodies, see below. Protein is produced in coli, insect cell, or mammalian expression systems, as desired.

Human embryonic kidney (HEK) 293 cells were transfected with 3 μg DC-STAMP DNA using LipofectAMINE (Gibco BRL). See Lanier, et al. (1994) J. Immunol. 153:2417-2428. 2 days after transfection, cells were harvested and used for Confocal Laser Scanning Microscopy (CLSM). Expression was checked by FACScan analysis in the FITC channel (Becton Dickinson & Co., Oxnard, Calif.) and usually 30-60% of the cells were positive for expression. In order to obtain a stable bulk population, G418 (1 mg/ml; Life Technologies Ltd, Paisley, Scotland) was added to the culture medium at day 2 after transfection. After 1 to 2 weeks, cells were sorted for GFP expression on the Coulter Epics Elite (Coulter, Hialeah, Fla.) and the resulting bulk population was used for CLSM. Cells were stained with rabbit polyclonal anti-GFP serum (kindly provided by E. Cuppen, Dept. of Cell Biology and Histology, University of Nijmegen, The Netherlands), either before or after cytospin preparations. Cytospins were fixed with acetone for 10 minutes, incubated with a horse anti-mouse biotinylated antibody and positive cells visualized by immunoperoxidase staining (Vectastain Elite ABC kit, Vector Laboratories, Burlingame, USA; AEC Substrate Kit, Zymed Laboratories, Calif.).

Confocal Laser Scanning Microscopy

Cells were attached to poly-1-lysine coated glass slides, after which GFP-fusion protein distribution was determined by Confocal Laser Scanning Microscopy (CLSM) at 488 nm with a krypton/argon Laser (Biorad 1000, Hercules, Calif.). The CSLM settings were: lens, 60×; gain, 1100-1350; pinhole, 1.5 μm; and magnification, 60×.

VIII. Isolation of Homologous Genes

The DC-STAMP or DSP-1 cDNA, or other species counterpart sequence, can be used as a hybridization probe to screen a library from a desired source, e.g., a primate cell cDNA library. Many different species can be screened both for stringency necessary for easy hybridization, and for presence using a probe. Appropriate hybridization conditions will be used to select for clones exhibiting specificity of cross hybridization.

Screening by hybridization using degenerate probes based upon the peptide sequences will also allow isolation of appropriate clones. Alternatively, use of appropriate primers for PCR screening will yield enrichment of appropriate nucleic acid clones.

Similar methods are applicable to isolate either species, polymorphic, or allelic variants. Species variants are isolated using cross-species hybridization techniques based upon isolation of a full length isolate or fragment from one species as a probe.

Alternatively, antibodies raised against human antigen will be used to screen for cells which express cross-reactive proteins from an appropriate, e.g., cDNA library. The purified protein or defined peptides are useful for generating antibodies by standard methods, as described above. Synthetic peptides or purified protein are presented to an immune system to generate monoclonal or polyclonal antibodies. See, e.g., Coligan (1991) Current Protocols in Immunology Wiley/Greene; and Harlow and Lane (1989) Antibodies: A Laboratory Manual Cold Spring Harbor Press. The resulting antibodies are used for screening, purification, or diagnosis, as described.

IX. Preparation of Antibodies Specific for Antigen

Synthetic peptides or purified protein are presented to an immune system to generate monoclonal or polyclonal antibodies. See, e.g., Coligan (1991) Current Protocols in Immunology Wiley/Greene; and Harlow and Lane (1989) Antibodies: A Laboratory Manual Cold Spring Harbor Press. Polyclonal serum, or hybridomas may be prepared. In appropriate situations, the binding reagent is either labeled as described above, e.g., fluorescence or otherwise, or immobilized to a substrate for panning methods. Immunoselection, absorptions, and related techniques are available to prepare selective reagents, e.g., exhibiting the desired spectrum of selectivity for binding.

X. Evaluation of Breadth of Biological Functions

DC-STAMP could possibly serve as a receptor for growth factors or hormones, which upon activation drive the differentiation into DC, or modulate DC function by directing T cell responses. Another possibility is a putative role for DC-STAMP as a receptor connecting the neuro-endocrine system to the immune system. Analysis of the effects of differentiation, maturation by various stimuli, and co-culture with T cells on the expression levels of DC-STAMP, will provide more insight into the specific function of this novel multimembrane surface receptor on DC.

Recently, DC pulsed with tumor antigens have been successfully used in vivo for the induction of anti-tumor T cell reactivity in melanoma patients. Nestle, et al. (1998) Nat. Med. 4:328-332. Thus, DC treated with these reagents may be useful in cell based therapies, e.g., cell transfer or in vitro cell treatments.

Biological activities of antibody to antigen are tested, based, in part, on the sequence and structural homology between the DC-STAMP and other membrane proteins. Initially, assays that show biological activities of 7TM receptors are examined. For the DSP-1, biological activities related to the function of monocyte, T, NK, and/or mast cells will be tested. Thus, assay for effects of polyclonal antibodies likely to contain antagonist antibodies affecting cells possessing the antigens will be tested. Primary assays include chemotaxis assays for the various cell types, e.g., monocytes, T, NK, and/or mast cells. Mast cell specific assays include, e.g., IgE mediated degranulation assays, mast cell chemotaxis assays, and effects on SCF/.IL-6 induced mast cell proliferation assays. Similarly, assays for effects on T cell, NK cells, or monocytes will be tested.

A. Effects on Proliferation/differentiation of Progenitor Cells

The effect on proliferation or differentiation of various cell types are evaluated with various concentrations of antibody. A dose response analysis is performed.

In particular, antibodies will be tested on cord blood cells to see if they have effects on proliferation or differentiation of early progenitor cells derived therefrom. Preferably, the cells are early precursor cells, e.g., stem cells, originating from, e.g., cord blood, bone marrow, thymus, spleen, or CD34+ progenitor cells. The antibodies will be tested for effects on myeloid and/or erythroid precursors, including B cell precursors.

B. Effects of Antibodies on Proliferation

Total PBMC are isolated from buffy coats of normal healthy donors by centrifugation through ficoll-hypaque as described (Boyum, et al.). PBMC are cultured in 200 μl Yssel's medium (Gemini Bioproducts, Calabasas, Calif.) containing 1% human AB serum in 96 well plates (Falcon, Becton-Dickinson, NJ) in the absence or presence of antibodies. Cells are cultured in medium alone or in combination with 100 U/ml IL-2 (R&D Systems) for 120 hours. 3H-Thymidine (0.1 mCi) is added during the last six hours of culture and 3H-Thymidine incorporation determined by liquid scintillation counting.

The antibodies would be tested for blocking signaling activity in many other biological assay systems, e.g., on T-cells, B-cells, NK, macrophages, dendritic cells, mast cells, hematopoietic progenitors, etc.

Antibodies are evaluated for effects in macrophage/dendritic cell activation and antigen presentation assays, T cell cytokine production or proliferation in response to antigen or allogeneic stimulus. See, e.g., de Waal Malefyt et al. (1991) J. Exp. Med. 174:1209-1220; de Waal Malefyt et al. (1991) J. Exp. Med. 174:915-924; Fiorentino, et al. (1991) J. Immunol. 147, 3815-3822; Fiorentino, et al. (1991) J. Immunol. 146:3444-3451; and Groux, et al. (1996) J. Exp. Med. 184:19-29. Antibodies will be tested for ability to affect mast cell degranulation, chemotaxis, etc.

Antibodies will also be evaluated for effects on NK cell stimulation. Assays may be based, e.g., on Hsu, et al. (1992) Internat. Immunol. 4:563-569; and Schwarz, et al. (1994) J. Immunother. 16:95-104. Other assays are applied to evaluate effects on cytotoxic T cells and LAK cells. See, e.g., Namien and Mire-Sluis (1998).

B cell growth and differentiation effects will be analyzed, e.g., by the methodology described, e.g., in Defrance, et al. (1992). J. Exp. Med. 175:671-682; Rousset, et al. (1992) Proc. Nat'l Acad. Sci. USA 89:1890-1893; including IgG2 and IgA2 switch factor assays. Note that, unlike COS7 supernatants, NIH3T3 and COP supernatants apparently do not interfere with human B cell assays.

C. Effects on the Expression of Cell Surface Molecules on Human Monocytes

Monocytes are purified by negative selection from peripheral blood mononuclear cells of normal healthy donors. Briefly, 3×10⁸ ficoll banded mononuclear cells are incubated on ice with a cocktail of monoclonal antibodies (Becton-Dickinson; Mountain View, Calif.) consisting, e.g., of 200 μl of αCD2 (Leu-5A), 200 μl of αCD3 (Leu-4), 100 μl of αCD8 (Leu 2a), 100 μl of αCD19 (Leu-12 ), 100 μl of αCD20 (Leu-16), 100 μl of αCD56 (Leu-19), 100 μl of αCD67 (IOM 67; Immunotech, Westbrook, Me.), and anti-glycophorin antibody (10F7MN, ATCC, Rockville, Md.). Antibody bound cells are washed and then incubated with sheep anti-mouse IgG coupled magnetic beads (Dynal, Oslo, Norway) at a bead to cell ratio of 20:1. Antibody bound cells are separated from monocytes by application of a magnetic field. Subsequently, human monocytes are cultured in Yssel's medium (Gemini Bioproducts, Calabasas, Calif.) containing 1% human AB serum in the absence or presence of antibodies.

Analyses of the expression of cell surface molecules can be performed by direct immunofluorescence. For example, 2×10⁵ purified human monocytes are incubated in phosphate buffered saline (PBS) containing 1% human serum on ice for 20 minutes. Cells are pelleted at 200×g. Cells are resuspended in 20 ml PE or FITC labeled mAb. Following an additional 20 minute incubation on ice, cells are washed in PBS containing 1% human-serum followed by two washes in PBS alone. Cells are fixed in PBS containing 1% paraformaldehyde and analyzed on FACScan flow cytometer (Becton Dickinson; Mountain View, Calif.). Exemplary mAbs are used, e.g.: CD11b (anti-mac1), CD11c(a gp150/95), CD14 (Leu-M3), CD54 (Leu 54), CD80 (anti-BB1/B7), HLA-DR (L243) from Becton-Dickinson and CD86 (FUN 1; Pharmingen), CD64 (32.2; Medarex), CD40 (mAb89; Schering-Plough France).

D. Effects of Antibodies on Cytokine Production by Human Monocytes

Human monocytes are isolated as described and cultured in Yssel's medium (Gemini Bioproducts, Calabasas, Calif.) containing 1% human AB serum in the absence or presence of antibodies. In addition, monocytes are stimulated with LPS (E. coli 0127:B8 Difco) in the absence or presence of antibodies and the concentration of cytokines (IL-10, IL-6, TNFα, GM-CSF, and IL-10) in the cell culture supernatant determined by ELISA.

Additional assays will be tested in the areas of bone remodeling, chondriocytes, neurons, adipocytes, gastrointestinal epithelium, or bronchial epithelium.

XI. Generation and Analysis of Genetically Altered Animals

Transgenic mice can be generated by standard methods. Such animals are useful to determine the effects of deletion of the gene, in specific tissues, or completely throughout the organism. Such may provide interesting insight into development of the animal or particular tissues in various stages. Moreover, the effect on various responses to biological stress can be evaluated. See, e.g., Hogan, et al. (1995) Manipulating the Mouse Embryo: A Laboratory Manual (2d ed.) Cold Spring Harbor Laboratory Press.

All references cited herein are incorporated herein by reference to the same extent as if each individual publication or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1-20. (canceled)

21. A binding compound comprising an antigen binding site from an antibody which specifically binds to a polypeptide consisting of residues 286-470 of SEQ ID NO:2.

22. The binding compound of claim 21, wherein:
(a) the antigen binding site is:
(1) specifically immunoreactive with the polypeptide consisting of residues 286-470 of SEQ ID NO:2;
(2) raised against a purified or recombinantly produced human DC-STAMP; or
(3) from a monoclonal antibody, Fab, or F(ab)₂; or
(b) the binding compound is:
(1) an antibody molecule:
(2) a polyclonal antiserum;
(3) detectably labeled;
(4) sterile; or
(5) in a buffered composition.

23. A detection kit comprising:
(a) the detectably labeled binding compound of claim 22;
(b) instructions for use; and
(c) a compartment for reagents.

24. The binding compound of claim 21 wherein the antibody specifically binds to residues 399-470 of SEQ ID NO:2.

25. The binding compound of claim 21 wherein the polypeptide is glycosylated.

26. The binding compound of claim 21 wherein the antibody specifically binds to residues 295-313 of SEQ ID NO:2.

27. The binding compound of claim 21 wherein the antibody specifically binds to residues 314-378 of SEQ ID NO:2.

28. The binding compound of claim 21 wherein the antibody specifically binds to residues 379-398 of SEQ ID NO:2.


SEQUENCE LISTING (9 sequences)

SEQ ID NO: 1 - DNA, 1960 bp, primate
  CDS (58)..(1467)
ggggggtggcatttctgcattcgaagaagaatctgagagaaacctgacgcagggagcatgggtatctggacctcaggcactgatatcttcctaagtctttgggagatttacgtgtctccaagaagccccggatggatggactttatccagcatttgggagtttgctgtttggttgctcttatttcagtgggcctcctgtctgtggccgcctgctggtttctgccatcaatcatagcggccgctgcctcctggattatcacgtgtgttctgctgtgttgctccaagcatgcacgatgttttattcttcttgtctttctctcttgtggcctgcgtgaaggcaggaatgctttgattgcagctggcacagggatcgtcatcttgggacacgtagaaaatatttttcacaactttaaaggtctcctagatggtatgacttgcaacctaagggcaaagagcttttccatacattttccacttttgaaaaaatatattgaggcaattcagtggatttatggccttgccactccactaagtgtatttgatgaccttgtttcttggaaccagaccctggcagtctctcttttcagtcccagccatgtcctggaggcacagctaaatgacagcaaaggggaagtcctgagcgtcttgtaccagatggcaacaaccacagaggtgttgtcctccctgggtcagaagctacttgcctttgcagggctttcgctcgtcctgcttggcactggcctcttcatgaagcgatttttgggcccttgtggttggaagtatgaaaacatctacatcaccagacaatttgttcagtttgatgaaagggagagacatcaacagaggccctgtgtgctcccgctgaataaggaggaaaggaggaagtatgtcatcatcccgactttctggccgactcctaaagaaaggaaaaacctggggctgtttttcctccccatacttatccatctctgcatctgggtgctgtttgcagctgtagattatctgctgtatcggctcattttctcagtgagcaagcagtttcaaagcttgccagggtttgaggttcacttgaaactgcacggagagaaacaaggaactcaagatattatccatgattcttcctttaatatatctgtgtttgaacccaactgtatcccaaaaccaaaattccttctatctgagacctgggttcctctcagtgttattcttttgatattagtgatgctgggactgttgtcctctatccttatgcaacttaaaatcctggtgtcagcatctttctaccccagcgtggagaggaagcgcatccaatatctgcatgcaaagctgcttaaaaaaagatcaaagcagccgctgggagaagtcaaaagacggctgagtctctatcttacaaagattcatttctggcttccagtcctgaaaatgattaggaagaagcaaatggacatggcaagtgcagacaagtcatgagagaccccgactactcctcagccacatcgcaccaacaattctcttcaggtctaggatggcagtcactattcatgccggataatagagaactatgtgacgcagtcctctcaggagtctgagtttacagagccaacttgcagcacctggttatgcctcctttcatctcaaagccaaagagctgccaggtaaatggttatgtggtctatgttccaaacaaaccacatgatcttgcctgtgtcacaatgtaacaagactctagctgggtcccctggtgatgagtttcagcatagaataatgttcaaggaaaagaaaacgaaaacagtttaaatctctaccacagcctcacaagcaaatgctaaggggaacatacatgtaaaaagccagcaaactatcttcaaactcttccgtccttaatgtcttccatggctattgcccccacaatggtctcttttctccctgctcccttattaaagaactctttctgaaaccc

SEQ ID NO: 2 - protein, 470 aa, primate
MGIWTSGTDIFLSLWEIYVSPRSPGWMDFIQHLGVCCLVALISVGLLSVAACWFLPSIIAAAASWIITCVLLCCSKHARCFILLVFLSCGLREGRNALIAAGTGIVILGHVENIFHNFKGLLDGMTCNLRAKSFSIHFPLLKKYIEAIQWIYGLATPLSVFDDLVSWNQTLAVSLFSPSHVLEAQLNDSKGEVLSVLYQMATTTEVLSSLGQKLLAFAGLSLVLLGTGLFMKRFLGPCGWKYENIYITRQFVQFDERERHQQRPCVLPLNKEERRKYVIIPTFWPTPKERKNLGLFFLPILIHLCIWVLFAAVDYLLYRLIFSVSKQFQSLPGFEVHLKLHGEKQGTQDIIHDSSFNISVFEPNCIPKPKFLLSETWVPLSVILLILVMLGLLSSILMQLKILVSASFYPSVERKRIQYLHAKLLKKRSKQPLGEVKRRLSLYLTKIHFWLPVLKMIRKKQMDMASADKS

SEQ ID NO: 3 - DNA, 1410 bp, Artificial Sequence
  Description of Artificial Sequencereverse translation
atgggnathtggacnwsnggnacngayathttyytnwsnytntgggarathtaygtnwsnccnmgnwsnccnggntggatggayttyathcarcayytnggngtntgytgyytngtngcnytnathwsngtnggnytnytnwsngtngcngcntgytggttyytnccnwsnathathgcngcngcngcnwsntggathathacntgygtnytnytntgytgywsnaarcaygcnmgntgyttyathytnytngtnttyytnwsntgyggnytnmgngarggnmgnaaygcnytnathgcngcnggnacnggnathgtnathytnggncaygtngaraayathttycayaayttyaarggnytnytngayggnatgacntgyaayytnmgngcnaarwsnttywsnathcayttyccnytnytnaaraartayathgargcnathcartggathtayggnytngcnacnccnytnwsngtnttygaygayytngtnwsntggaaycaracnytngcngtnwsnytnttywsnccnwsncaygtnytngargcncarytnaaygaywsnaarggngargtnytnwsngtnytntaycaratggcnacnacnacngargtnytnwsnwsnytnggncaraarytnytngcnttygcnggnytnwsnytngtnytnytnggnacnggnytnttyatgaarmgnttyytnggnccntgyggntggaartaygaraayathtayathacnmgncarttygtncarttygaygarmgngarmgncaycarcarmgnccntgygtnytnccnytnaayaargargarmgnmgnaartaygtnathathccnacnttytggccnacnccnaargarmgnaaraayytnggnytnttyttyytnccnathytnathcayytntgyathtgggtnytnttygcngcngtngaytayytnytntaymgnytnathttywsngtnwsnaarcarttycarwsnytnccnggnttygargtncayytnaarytncayggngaraarcarggnacncargayathathcaygaywsnwsnttyaayathwsngtnttygarccnaaytgyathccnaarccnaarttyytnytnwsngaracntgggtnccnytnwsngtnathytnytnathytngtnatgytnggnytnytnwsnwsnathytnatgcarytnaarathytngtnwsngcnwsnttytayccnwsngtngarmgnaarmgnathcartayytncaygcnaarytnytnaaraarmgnwsnaarcarccnytnggngargtnaarmgnmgnytnwsnytntayytnacnaarathcayttytggytnccngtnytnaaratgathmgnaaraarcaratggayatggcnwsngcngayaarwsn

SEQ ID NO: 4 - DNA, 942 bp, primate
  CDS (1)..(939)
  mat_peptide (64)..(939)
atggccttaccagtgaccgccttgctcctgccgctagccttgctgctccacgccgccaggccggattacaaggacgatgacgacaagatcgatctgagcaaatgcaggaccgtggcgggccccgtggggggatccctgagtgtgcagtgtccctatgagaaggaacacaggaccctcaacaaatactggtgcagaccaccacagattttcctatgtgacaagattgtggagaccaaagggtcagcaggaaaaaggaacggccgagtgtccatcagggacagtcctgcaaacctcagcttcacagtgaccctggagaatctcacagaggaggatgcaggcacctactggtgtggggtggatacaccgtggctccgagactttcatgatcccgttgtcgaggttgaggtgtccgtgttcccggcatcaacgtcaatgacacctgcaagtatcactgcggccaagacctcaacaatcacaactgcatttccacctgtatcatccactaccctgtttgcagtgggtgccacccacagtgccagcatccaggaggaaactgaggaggtggtgaactcacagctcccgctgctcctctccctgctggcattgttgctgcttctgttggtgggggcctccctgctagcctggaggatgtttcagaaatggatcaaagctggtgaccattcagagctgtcccagaaccccaagcaggctgccacgcagagtgagctgcactacgcaaatctggagctgctgatgtggcctctgcaggaaaagccagcaccaccaagggaggtggaggtggaatacagcactgtggcctcccccagggaagaacttcactatgcctcggtggtgtttgattctaacaccaacaggatagctgctcagaggcctcgggaggaggaaccagattcagattacagtgtgataaggaagacatag

SEQ ID NO: 5 - protein, 313 aa, primate [sequence omitted]

SEQ ID NO: 6 - DNA, 603 bp, primate
  CDS (1)..(600)
  mat_peptide (64)..(600)
atggccttaccagtgaccgccttgctcctgccgctagccttgctgctccacgccgccaggccggattacaaggacgatgacgacaagatcgatatgacacctgcaagtatcactgcggccaagacctcaacaatcacaactgcatttccacctgtatcatccactaccctgtttgcagtgggtgccacccacagtgccagcatccaggaggaaactgaggaggtggtgaactcacagctcccgctgctcctctccctgctggcattgttgctgcttctgttggtgggggcctccctgctagcctggaggatgtttcagaaatggatcaaagctggtgaccattcagagctgtcccagaaccccaagcaggctgccacgcagagtgagctgcactacgcaaatctggagctgctgatgtggcctctgcaggaaaagccagcaccaccaagggaggtggaggtggaatacagcactgtggcctcccccagggaagaacttcactatgcctcggtggtgtttgattctaacaccaacaggatagctgctcagaggcctcgggaggaggaaccagattcagattacagtgtgataaggaagacatag

SEQ ID NO: 7 - protein, 200 aa, primate
MALPVTALLLPLALLLHAARPDYKDDDDKIDMTPASITAAKTSTITTAFPPVSSTTLFAVGATHSASIQEETEEVVNSQLPLLLSLLALLLLLLVGASLLAWRMFQKWIKAGDHSELSQNPKQAATQSELHYANLELLMWPLQEKPAPPREVEVEYSTVASPREELHYASVVFDSNTNRIAAQRPREEEPDSDYSVIRKT

SEQ ID NO: 8 - DNA, 939 bp, Artificial Sequence
  Description of Artificial Sequencereverse translation
atggcnytnccngtnacngcnytnytnytnccnytngcnytnytnytncaygcngcnmgnccngaytayaargaygaygaygayaarathgayytnwsnaartgymgnacngtngcnggnccngtnggnggnwsnytnwsngtncartgyccntaygaraargarcaymgnacnytnaayaartaytggtgymgnccnccncarathttyytntgygayaarathgtngaracnaarggnwsngcnggnaarmgnaayggnmgngtnwsnathmgngaywsnccngcnaayytnwsnttyacngtnacnytngaraayytnacngargargaygcnggnacntaytggtgyggngtngayacnccntggytnmgngayttycaygayccngtngtngargtngargtnwsngtnttyccngcnwsnacnwsnatgacnccngcnwsnathacngcngcnaaracnwsnacnathacnacngcnttyccnccngtnwsnwsnacnacnytnttygcngtnggngcnacncaywsngcnwsnathcargargaracngargargtngtnaaywsncarytnccnytnytnytnwsnytnytngcnytnytnytnytnytnytngtnggngcnwsnytnytngcntggmgnatgttycaraartggathaargcnggngaycaywsngarytnwsncaraayccnaarcargcngcnacncarwsngarytncaytaygcnaayytngarytnytnatgtggccnytncargaraarccngcnccnccnmgngargtngargtngartaywsnacngtngcnwsnccnmgngargarytncaytaygcnwsngtngtnttygaywsnaayacnaaymgnathgcngcncarmgnccnmgngargargarccngaywsngaytaywsngtnathmgnaaracn

SEQ ID NO: 9 - DNA, 600 bp, Artificial Sequence
  Description of Artificial Sequencereverse translation
atggcnytnccngtnacngcnytnytnytnccnytngcnytnytnytncaygcngcnmgnccngaytayaargaygaygaygayaarathgayatgacnccngcnwsnathacngcngcnaaracnwsnacnathacnacngcnttyccnccngtnwsnwsnacnacnytnttygcngtnggngcnacncaywsngcnwsnathcargargaracngargargtngtnaaywsncarytnccnytnytnytnwsnytnytngcnytnytnytnytnytnytngtnggngcnwsnytnytngcntggmgnatgttycaraartggathaargcnggngaycaywsngarytnwsncaraayccnaarcargcngcnacncarwsngarytncaytaygcnaayytngarytnytnatgtggccnytncargaraarccngcnccnccnmgngargtngargtngartaywsnacngtngcnwsnccnmgngargarytncaytaygcnwsngtngtnttygaywsnaayacnaaymgnathgcngcncarmgnccnmgngargargarccngaywsngaytaywsngtnathmgnaaracn